SEQUENCE LISTING (79 sequences)

SEQ ID NO: 1 - protein, 261 aa, Akkermansia muciniphila
MNMERKTGLMNKKYVSPCFLPGMRLGNIMFTLAAACAHARTVGVECRVPWAYNDASLMLRSRLGGWVLPSTPCGTNEPPSWQEPSFAYCPVPSRIRTGGLRGYFQSARYFEGQEAFIRALFAPLTAEKEPGAVGIHIRLGDYRRLRDKHRILDPGFLRRAAGHLSSGKNRLVLFSDEPDEAAEMLARVPAFGRFALEIDRGAPCESLRRMTAMEELVMSCSSFSWWGAWLGNTRKVIVPRDWFVGGVEDYRDIYLPHWVTL

SEQ ID NO: 2 - protein, 297 aa, Porphyromonas catoniae
MKRIYLSIYGGLGNQLYILAYADYLQRMLGTRPYLLNELQRTKADTSSLDRTRRDLFSELIAYLGFQFVDTDSREFRLLKKWERHIKHYQEEPNKHGIYLQNILPPLQENHRWTLPLVCRVSGYFQSCHYVGTDFRMRVSKFLERHATSADLVREYTSMIQPEDVAIHLRRGDFVALQHTGIQLFGAEHYTKGLALQAQQQPIGRVFVFSDDFEAIGEELSLLANNYQLVLVKGLTPLQDLFLLTCFRRYVLANSTFSWWGALCSKYGDAVKVVVPKKPLLISYPEDSYFPPSWEQI

SEQ ID NO: 3 - protein, 311 aa, Helicobacter sp.
MFSVRLMGGLGNQMFIYAFAKAIKAQGYPVRLFYYDTDYNVPQTHNIRNLEIVDFGIAMCIETMCYEEPQIKKSFFERALGFIKRKLKIHSPHSSSLISDHCEIALTKDFLDTLNPNAMFNGYFQNVVFFDHLRESLLRDFTLKRPLTPANEALKHQILQTPNSCFLHIRRGDYLQIPIYVKLGSTYYNNAIKALKDKISKPHIFVFSNDIAWCKEFFLDSLDPLVIENVTFSFIENNDEGNAIEEMELMRSCQHAIIANSTFSWWAAYLIDSAQKLCIMPKHFFNDPQQEVAHKLIPPPLHSLSQTIVIG

SEQ ID NO: 4 - protein, 289 aa, Capnocytophaga canis
MMRHYICLQGGLGNQLYILGYAFYLQEQGCKNICLFYEQKQNGDTVDTQKRNIIDDLPKELGFKSLYISSLWLKVLRNLCKLPMISKLVDYYEEPTEEWAVFTPFSPLRKKRVSVHIGYYQSFYYQTPLFIERLKRFFFKNVLLERFSPVENDAAIHIRRGDFLTGVNTMIYSEIGVKYYLEGLEKLNREQGIGKIYVFSDDFQAITNDIEEISKIYTVELMQGNSVLQDIRMLMCFKRYVLGNSTFAWWGAKLSEKQNPIVVVPATPWKINLKKEVTPYPKDWILLEN

SEQ ID NO: 5 - protein, 289 aa, Capnocytophaga leadbetteri
MKANYVLFQGGLGNQLYQLAYTDFLKRNGYSNVKLITPSNKNKGDTKDKNKRPLITQLPEKIGIDCVDFGHKYFYSFLQRLPKFPLYKSFLRRLINVEKEPPYQWAIFHPITREKYRLNIHIGYYQSNLYISDSFKQQVAKVIESLSPNIKFSITNNDVAIHIRRGDFLIGNNASVFNKIELPHYLQGLTILSERMNIQKVYIFSDDFEAIKEDIKTIAENYEVVLVEGQSVLADFALLQKFTNFVIGNSTFAWWGAMLANASNVIVPKKPWKIEMENMSPYPDNWTTI

SEQ ID NO: 6 - protein, 303 aa, Helicobacter sp.
MFSVRLMGGLGNQMFIYAFAKAIKAQGYPVRLFYYDTDYNVPQTHNIRNLEIVDFGLNLPIQRLCSKGIHRNVKRIIAFIHRKIAKYLFTFVSDCHNVSPTKDFLDTLNPNAMFNGYFQNVVFFDHLRESLLRDFTLKRPLTPANEALKHQILQTPNSCFLHIRRGDFLPLPEYIQLDSTAYYNNAIKALKDKISKPHIFVFSNDIAWCKEFFLDSLDPLVIENVTFSFVENNDEGNAIEEMELMRSCQHAIIANSTFSWWAAYLIDSAQKLCIMPQRFYGIQQEENCDIPPPPHTLAVYSKN

SEQ ID NO: 7 - protein, 288 aa, Kingella denitrificans
MAKHYIHFANGLGLGNQLYLLAYTDYLKSLGADASLFLMGNGQVGDTKDKAKRNLVLEIPQRLGMAVVDKKQISRSFRYIFCKAFYGKEKLNWREPKDQWAVFFEGVHGKYPINFYYGYFQSHHYIAEPFKQRLKTVLRALKPVDVAVGENDVALHIRRGDFLNPENAGLIRTVGLDYYLNGLDYIRQRQTIGTVYIFSDDFAAIEKEIAAISADYPVRLMQGQSVLEDMNWLTYFKHYVVGNSTFAWWGCLLSDHQDGLVVVPQRPWVQEQPAASQYLPHWVQLPNE

SEQ ID NO: 8 - protein, 288 aa, Pseudoalteromonas distincta
MIKVKAIGGLGNQLFQYATARAIAEKRGDGVVVDMSDFSSYKTHPFCLNKFRCKATYESKPKLINKLLSNEKIRNLLQKLGFIKKYYFETQLPFNEDVLLNNSINYLTGYFQSEKYFLSIRECLLDELTLIEDLNIAETAVSKAIKNAKNSISIHIRRGDYVSNEGANKTHGVCDSDYFKKALNYFSERKLLDEHTELFIFSDDIEWCRNNLSFDYKMNFVDGSSERPEVDMVLMSQCKHQVISNSTFSWWGAWLNKNDEKVVVAPKEWFKSTDLDSTDIVPNQWIKL

SEQ ID NO: 9 - protein, 297 aa, Porphyromonas catoniae
MKRIYLSIYGGLGNQLYILAYADYLQRMLGTRPYLLNELQRTKADTSSLDRTRRDLFSELIAYLGFQFIDTDSREFRLLKKWERHIEHYQEEPNKHGIYLQNILPPLRENPRWTLPLVCRISGYFQSYHYVSTDFRRRVGKFLNLHATSADLAREYALMIQPEDVAIHLRRGDFVALQHTGVQLFGAEHYAKGLALQAQQQPIGRVFVFSDDFDAIQEELGLLADNYQLVLVKGLTPLQDLFLLTCFRRYVLANSTFSWWGALCSKYGNEVKVVVPKKPLLISYPEDSYFPPSWEQI

SEQ ID NO: 10 - protein, 281 aa, Campylobacter mucosalis
MIIVKIVGGLGNQMFCYAYAKMLSIGGYNVKMDTSVYKTYKLHGGYQLDKYNIDLATLSGNKNAKFYSNGVVFKILKKFGLLKIKREESLLFDESFKTPNDKVYIEGYFQSEKYFCDIKDILLNQFVLKTTLSNYAISIKDKITQNSCSIHIRRGDYLLGKNINYHGVCDLKYYQDATDFLEQKCGDLTYFIFSDDIEWAKGNLKLNNAFYVDSSDRLPHEDIYLMSLCQNNIIANSTFSWWGAYLNQNQNKIVIAPKIWFSNAKLQSQTSDLIPNEWIRV

SEQ ID NO: 11 - protein, 283 aa, Pedobacter kyungheensis
MIYVVFSDRLGNNLFQMAAALSLSEKITICVPLQEEYSATLKYKDTFFKGFDILNYIPDGIPVYEEPFYHYNSVPYTEGTDLIIKGYFQSHRYFDRSLVLKQYSIDDNTIFFIEKKYPEILKGGYTSIHIRRGDYLKMLYKHPFCGLKYYKKAIDLIGKNENFIIVSDDITWCKNNIKLKNVIFAENTSPIIDLYIQSYCENNILSNSSFSWWGAYLNKHINKKVIVPSLWFGYKANFSTKDLLPRDYIVIINKYSFVGYLKSVMQLVKNKIFFLLNNKDVQN

SEQ ID NO: 12 - protein, 286 aa, Noviherbaspirillum autotrophicum
MIVVRLIGGLGNQMFQYASALALAKRTGSDLILDLSAFKHYSLRDYGLQQWKISARIASSVELRKFPRWGARVARYTQKLGIKTRFYSELGLGFDPAFMKLHAPVYLNGYFQSEKYFLNIRSILLEEFQPSNPLLAQNSRHASDAIESNSVAIHIRRGDYVSNTHNLSIHGVCSLGYYERAINIIRSKIGPANFFVFSDDMEWVRENIRIPDEVVFVEGNNARPEADIHLMSLCKHNICANSSFSWWGAWLNRNPEKIVIAPDPWFVSKALDSNHLIPQDWFQVSS

SEQ ID NO: 13 - protein, 294 aa, Microbacterium trichothecenolyticum
MTVRDGVCAYVMGGLGNQLFILAAAWEQSRRLNVPLYLDRSHFAVGGTFAYGLDAVPNPGHVLSPTESPWRSVRINRERVLPLPRRPWGRVYLERDGDTYSPAINGIRPGTTLVGYFQSPRYFPTVRDELASAMLKTEETAEETARIESMLAAPAITLHLRRGDYLAVSADRQFIASVDYAVRGVRHLRSMGLDHPVRVFSDSVDLVKDELRGVEGDFEFVEDDGVLGTWATLKAMSAGTAMIMSNSSFSWWGAELMRGRLGPGVPVIAPRPWTQTGTAKADLLEPAWVTLDAR

SEQ ID NO: 14 - protein, 272 aa, Candidatus symbiothrix dinenymphae
MNVVQLQGGLANQMFQYAFGQALGEDTLYDVSWFERAKSERTTSRPFCLDVFNIPATVFVNRRPPLLARLVGKYKVVRERNWDEFQPEFLNLGGRGTNYFKGYFQNERYLNCVKDKISADFSLKNTMNSDNLELLKQIRNCNSVSLHIRRGDYVKQPDYHPLCGIEYYQAAMKMIEGHYFLFSDDIEWCKANAKTDMPMTIVDINSAETGYWDMELMKNCKHNIIANSSFSWMAAYLNPNPDKMVIAPKDWNKHNGEAISNRGLSEKYIQIK

SEQ ID NO: 15 - protein, 275 aa, Pedobacter sp.
MIGISIQCRLGNQLFQYAFIISLSKTLNSKYFISEKIERFTLPDYFELPHYNPNLNLLYKIILKIKKGYLRKPLQNYSLTRNNIARDNEIYVGYFQSETFFNQLKNDIDSFIKVRKEYKQVFSNQYGNFFSEHKTIAIHLRRGDYLNLDNWWLENLGSNNLTLPLDYYKNCFAAIENLHHYKLMFISDDIEFVKSEFGYLENAEFHNNELIIDFQIMMNADICIVSNSSFAWWAAYLNPKATKKILCPKYWLGFNIKKEYPKNIIPKDWKQIIAK

SEQ ID NO: 16 - protein, 288 aa, Chryseobacterium sp.
MTITKLQGGLGNQMFQYAVARSRCVDEKIYLDFSFLQKNNVSTEHFTKRDFELGVFGIQYKEFSDRERKICFGTSTKDKILRKLFYGRTETVHQIENEFVIIPAAQNIYFDGYFQSEKYFASIRNQLLTEFTFPELDGANIRILDQINSVQNSVSIHIRRGDYLKPEVLKYHGSLGENYYKAGLELLKAKFPNLYYFVFSDDISFAKTLFKDLENTYFVNINSGKDSWKDMFLMAQCRHHIIANSSFSWWGAWLSQKQGINIAPENWFNKEVANFDINNIVPSDWITI

SEQ ID NO: 17 - protein, 283 aa, Microbacterium sp.
MGGLGNQIFILAAAWEQARRLDVPLYLDASHFSVGGTFRYGLDAIDHPGRLLGPKESPWRSVRINRERVLPFPRRPWGHIFLERDGDRYQPAIEQITSGTTLVGYFQSPRYFPNVRDQLIASMLDTADTEAEASRLREYDRTPAISLHLRRGDYLAVSADRQFIASVAYAVRSVKLLRAMGIDLPVRVFSDSVDLVKDELRGVEGDFDFVEDDGALGTWATLKAMSAGHAMIMSNSSFSWWAAELMHRRSGGRAPVIAPRPWTQTGTAKSDLLHADWITLDAR

SEQ ID NO: 18 - protein, 264 aa, Ramazzottius varieornatus
MFIYASLWGIAKKNGLTPTIPFTKLHDAFHLDLSPEQFFDLSDCDNYVFGMDECCLYDERTEQIFQRAVGRNVSLWGYFQSSRYFHPEHETDIRRQFAFKENIDVRANDIIKGFRLRSGDSMQKRPSVGIHIRRGDVLVKSFWRNFGFTVATREYFRHAMEYLKNKGVIDPIFLVCTNDVEWSRQNLAGFDVHFVENQTSHVDMAILTHMNHLILSAGTFSFFAGYLSSAQHIIYYKGWPRPGSKYAGMMDLSSFWLPEWIAME

SEQ ID NO: 19 - protein, 288 aa, Candidatus wolfebacteria
MIIIKLKGGLGNQMFQYACAKHLAERNGDVLKLDISLYQPGNAPAGDTARSYALGAFSISAQVASKEEVRRVRGLLWHIMALAMKVINRVRPISSYMFDPQVLERRGNVYLDGYFQSERYFKDIKACIRNEFRLREPMGEQAQAMLRDIDGSDSVSLHIRRGDYVTNKNANAFHGTCPPEYYHAAIKEIGKHVHSPKFFIFSDDIEWAKADVGMPAHATYVSRDGIADYEELLLMSKCKHNIIANSSFSWWGAWLNANSNKIVVAPKRWINDERVDTSDAVPVEWVSM

SEQ ID NO: 20 - protein, 272 aa, Candidatus yanofskybacteria
MITFRKLGLLGRFGNQLFQYAGARMYADINGFKSAFPGWVGNKIFENIISYNNREYLLSRFLPTRQLNDFLSYTRTDKIKYMLRLQKQLPQTIALNKLYAHPEDNINFLGYFQNELSLKILKERKNYVLGWFVFKKDIADEFKKVTQNYKPWTGIHIRRGDFVKRGLSLPVFLYKDFLKSVDKKNIYISCDDPETISEFKDYSLIRPANPLPEIPNFIFDFWMLKESEMVLGCGSTFSWWAAYLGNRNNYFSPPLTHLWPKGYKPTLGKMDI

SEQ ID NO: 21 - protein, 276 aa, Spirochaetes bacterium
MIGIKIGGGLGNQMFQYAFGYVVAKKFSTSFYLEKRNQFKLDKYFSLRILENSINSLIKPLYKMQTKLGKIKMYDWEDFGTKTPESFISQIKDNIYYCGYFQSEKYFEGFYKDLQKIFKIKKEYIEQFNSKYEKFYKENKVIAIHIRRGDYINFGDDSLGGKNMTLPISYYTNCLKQIKDISSYKIIFISDDIEFVKKEFGQKENYFFESNNEIIDFQVILNADKVIMSNSSFSWWASYLNIKATDIYCPNYWLGFKIGTENPIGIVCKKWTRLDI

SEQ ID NO: 22 - protein, 296 aa, Pedobacter soli
METEIPVTQDLDQNKQVYLITMVGISIQCRLGNQLFQYAFITALSKKLNTKYFISEKIERFTLPDYFELPQYSSKLNFLRKLILKIKKGYTLKPLQHYEITQNTPAKDNEIYVGYFQSESFFEGTKDNIGELIKVRPEHKNAFNNQFEKTFSQQKTIAIHLRRGDYLNLDDWWLENLGGNNLTLPITYYEKCFAAIKNLNNYRLLFISDDIAFARLNFGHLENAEFHNNELIIDFQIMMNADICIVSNSSFAWWAAYLNAKSTKKIFCPEYWLGFNINTEYPKNIIPDSWEQIAVK

SEQ ID NO: 23 - protein, 304 aa, Bacteroidales bacterium
MENNILQTTLLGRLGNQLFIYAATRVMALESGKKLKLTCDAMERFRATYRLDCFNIPQDQVSLVSDKHLSIKQKIGYFFYLLKCRHKTVVQIHELENKYQKFYHRFGLILNQEGYIKPLLLEGGNWYALGYFQSDKYFTKYEDLIRKELTFKTDMFCKDSKMLGSKLRANKNSVCLHIRRGDYLNDPVFAVCDINYYNKAIDKLLELIPNAEFFVFSDSIDEVREFLAKYCDNYFHYIDVKYTDQESLYLGSCCNNFIMTNSTFSWWMQYLSDNPNKIVLAPNRWYNTNNPCDIYQDNWMIIEV

SEQ ID NO: 24 - protein, 292 aa, Butyrivibrio sp.
MIIIKMQGGLGNQLFLYGLYKELLYLGREVKMDNISGFEDDPQRSPSLYKLGISYDIAEDGEIRTMRDSYMDPLSRIRRKITGRKNKDYYEALDGNFDEHVLEADDIYLNGYFQSDDYFKDDEVRQELIADICKNKDLYLGDQDVTDIRNAIRSSNSVSMHIRRGDYLTPVSRKTYGGICTDDYYAKAMELVRSSYPDAQFYIFSNDIDWCRAKYKDTDNISFVNISGDDSDIMEFFLMSECRHHILANSSFSWWAAFLGSADDRDRLNIVPSKWTNTRQMKDIYASWMTRI

SEQ ID NO: 25 - protein, 309 aa, Butyrivibrio fibrisolvens
MIIIKLQGGLGNQLFLYGLYRNLERLGRDVKLDIESGFECDELRSPCLDGMKLKYEVATRKEVIAIRDSYMDILSRIRRKITGRKTFDYYEPEDGNFDPKVLELTHAYLNGYFQSEKYFGDDNSAKALKIELTTNRYDILSSSDLVTDIYNDIRKSESVSLHIRRGDYLTPGIIETYGGICTDDYYDKAIAMIRDKHPDAKLFVFSNDIDWCKEKMADYKNISFVSTMPVNNIIEEVFNSSQSSKITNNYKDLAELYLMSACKHHVLANSSYSWWGAWLSDNEGMTIVPSKWLNNKNMTDIYTRDMILI

SEQ ID NO: 26 - protein, 277 aa, Candidatus magasanikbacteria
MIFDKLSGGLGNQMFQYAAGYSLSLNNRIPLNLDLSSFEHKKTGITHRYFLLNKFNIDRNILINHMEKISGYRKFLSKFITKFFGENFYYNITFLSSKYLDGYFQSEKYFKNIEDILRKEFTLKNEMSVVARQVESKISNSINSVSLHIRRGDYVLDNKTNSYHGICDLDYYKKAVEYFKNKLGELNIFVFSDDIVWVKENLRFENLYFVSSPDIKDYEELILMSRCKHNIIANSSFSWWGAWLNANKNKTVITPKKWFQKYNINQKHIVPKSWIRL

SEQ ID NO: 27 - protein, 292 aa, Butyrivibrio fibrisolvens
MIIIKMQGGLGNQLFLYGLYKELLFLGREVKMDNNAGFIEDPLRDPVLSKLGISYDLAEDKEIRKMRDSYMDPLSRIRRKLTGRKNKDYYEALDGNFDETVLKADDIYLNGYFQSEKYFNDDSVRQELRADICKNKERYMSESDVADLRQEIANANSVSMHIRRGDYLTPVSKETYGGICTDEYYEKAMDLVKEKNPDARFYIFSNDIDWCKEKYKDTDNVSFVSLTGDDSDIKEFFLMSECRHHILANSSFSWWAAFLSAADDNKSLNIVPSKWTNTRQMKDIYASWMTRI

SEQ ID NO: 28 - protein, 293 aa, Chryseobacterium scophthalmum
MVAVELIGGLGNQMFQYATARALALHRDEPLSIDGRLFDNYKLRNFCLSHFSIEAIVVKNDLPLKTPGFSKKVVDQILKKTNTFILQNKLFSVYQEKNLLFDDSLFRNGKKNIYLKGYFQSEKYFVKYEDQLRKDFKIVTPLKKETIDLLKIIKEKNSVSLHIRRGDYVSNPTVNAIHGTCDLNYYHRAIEIVKEKILHPVFFIFSDDIDWAKENLKLENTTYFVDFNDASTSYEDLKLMSACKHNIIANSSFSWWGAWLNTNKSKTVIAPSKWFNTDVLNSQDIIPESWMKI

SEQ ID NO: 29 - protein, 261 aa, Akkermansia sp.
MNMERKTGLMNKKYVSPCFLPGMRLGNIMFTLAAACAHARTVGVECRVPWAYNDASLMLRSRLGGWVLPSTPCGTNEPPSWQEPSFSYCPVPSRIRTGGLRGYFQSARYFEGQEAFIRALFAPLTAEKEPGAVGIHIRLGDYRRLRDKHRILDPGFLRRAAGHLSSGKNRLVLFSDEPDEAAEMLARVPAFGRFALEIDRGAPCESLRRMTAMEELVMSCSSFSWWGAWLGNTRKVIVPRDWFVGGVEDYRDIYLPHWVTL

SEQ ID NO: 30 - protein, 263 aa, Lingula unguis
MFQFASLLGIAARNNLTAVIPYNPEMERIFKISTPSTKNLQNTLGMYLTVTQFYGRCCYYDILTETLDPRHNYLLYGYFQSWKYFKNVNDIVRQEFQFREEILNEAKKFHRDVSNKFQCHENNSCTFVGIHIRRGDISDYDVYKKAGYTTPSEQYFKTAMNFFKRKHKNKVVFIVCSDSSKGWQKKLTSSDVVFSHAKDQGVDLAILSLCQHMIISTGTYGWWAAFLTGGTTVYYRDWPKAGSWLYDQVFKEDYFVRGWIPMK

SEQ ID NO: 31 - protein, 263 aa, Lingula unguis
MFQFASLLGIAARNNLTAVIPYNPEMERIFKISTPSTKNLQNTLGMYLTVTQFYGRCCYYDILTETLDPRYNYLLYGYFQSWKYFKNVNDIVRQEFQFREEILNEAKKFHRDVSNKFQCHENNSCTFVGIHIRRGDISDYDVYKKAGYTTPSEQYFKTAMNFFKRKHKNKVVFIVCSDSSKGWQKKLTSSDVVFSHAKDQGVDLAILSLCQHMIISTGTYGWWAAFLTGGTTIYYRDWPKAGSWLYDQVFKEDYFVRGWIPMK

SEQ ID NO: 32 - protein, 273 aa, Flexibacter sp.
MIVVQLIGGLGNQLFQYAAAKAFALETEQKLYIDVSQFESYKLHNYALNHFNIISNVYNKPNRYFKKIKSFYQKNTSYKEVDFGYNQDLFNLKGDTIFLDGYFQSEKYFKKYEKEIREDFEIRTPLKKITTDTIAKIETVNSVSIHIRRGDYLNNPLHNTSKEEYYKKALEIIEEKITNPVFFVFSDDINWVKSNFSTNHETVFIDFNDASTNFEDLKLMASCKHNVIANSSFSWWGAWLNKNQNKIVIAPKLWFNDNSININDIIPTSWLKI

SEQ ID NO: 33 - protein, 295 aa, Bacteroidetes bacterium
MIIVNLYGGLGNQLFQYACGKAVAERLGVELQLDISHLKEYGKATNFTVRDFELGVFSINEKTADIQEVRKYVPNLYKTSRYYHQLWRIKRLFNGRHLFIERTWQRDRYIENIEKVKDNTYLYGYFQSARYFADIEEKLRTTLVLKDRIDAENEALIQQMENENSVSIHIRRGDYENSRFSLPELDTYYLPAIKAIQTQIPQPKFYIFSNDAEWVKKNFNSLEIKNEIVSINSGSRSFMDMILMSRCKHNIIGNSSFSWWGAWLNQNPDKMIVVPKNWYKNGRETDLQLKEWIKI

SEQ ID NO: 34 - protein, 279 aa, Pedobacter sp.
MIIVKLQGGLGNQMFQYAAARTLSNSGKVYLDFSFLNKNNITTDSFTARQFELDIFKRTKTHISNPYFIRLIRSNRKIFKMLSPRYQEVKDENIFDFLNNNSSNLYLDGYFQNPLIFQDIRNILMDEFTFPELSKLSKEIELNILSTTNPVAIHIRRGDYTKPEIQSYHGILPLNYYKQGIERIKSKVENPIFYIFSDDPEWCEHNLSFIDNKHIIAGTKQAWEDMFLISKCKHQIIANSSFSWWGAWLNANPEKIIVAPKKWFNNVDINILPKAWISL

SEQ ID NO: 35 - protein, 317 aa, Candidatus planktophila lacus
MKRSNSSVGVSLTGGLGNQLFQVAAALAMSEGERVTLISSYGKPRFSKNNEAEIYSLIRAEDWFERDDEVSSWLPAKCVGYILRMGVQPKIWEKGLVVFFINKIANLILSISRNSKIRILAGKGVGYFTLPEKKYFTLLSGYFQSYRWASLDDVYKKLFHLHPKHPGSDLKYYESLTSDCKPLVIHIRLGDYLSERDFGIPSVEYYKSAVSRISTEVEFDEIWVFSDTLTKAKEILRFDGRFKVRWIGELDGSAASTFQAMRHGAGYIIGNSTFSWWAAFLRLDTKAPVIAPKPWFRGMEEPFELIPKNWTRIDSNY

SEQ ID NO: 36 - protein, 304 aa, Rhizobiales bacterium
MATQPRIPPDQTERKTQVAVQLHGGLGNQMFQAAAGLALAERLGASLLFDTSRFRDKGLRAYALSAFALSAKVLHETPTPLAALRRAILKGMGRKSATQPNYWRGRFYREPHFHFDPGFTHLAGDTMIAGYFQSPHYFAGYESHIALVLAPEKLMSNAALHLAEILNGEASVAIHLRRGDFAADPKAAAVHGVLDWSYYDRAVAHIRAQQPEARFFVFSDNPEAAAEGAARWPNAEPMAGTSAGDDLFLMSRARHHIIANSSFSWWSCWLDRREGGIRIAPEQWFAAGNPQETRDLCPPEWLRL

SEQ ID NO: 37 - protein, 396 aa, Helicobacter sp.
MIEIRLQGRLGNQFFIYAFAKNLRKHLRNSNSPKLAKQEVILVCDDIHSYEVPDILKYNVDKSFFRPSAFHGFKTTIDKAFFFMFRVARKGIYLITKKTISYRHKNKEVPHMLPETLMETYENGEFDKAVIAKCLNKFIIGYFQSELYFEDVKEELKKDFSLQENHLPVMKVSAFKALLTKIKEQDSIFLHIRRGDYLSSVNVTKYAQLGVNYYTKAIALVLQKCPNAKFYIFSDDVAWVKKEGISLFGLNKLDYEVVNLNSPNAGYLDLELMKACKGAITANSSFSYWGAYLMENPKVVIAPFPFHFIHSNMDLLPRDFIVLDCKTGEVLEEKDYMYTNSKSPKSLKVSAFTEMFEAAFDVVYNPNSGGGGGNIPLQKTTKIAKVVNFFKKLILR

SEQ ID NO: 38 - protein, 277 aa, Candidatus magasanikbacteria
MIFDKLSGGLGNQMFQYAAGYSLSLNNRIPLNLDLSSFEHKKTGITHRYFLLNKFNIDRNILINHMEKISGYRKFLSKFITKFFGENFYYNITFLSSKYLDGYFQSEKYFKNIEDILRKEFTLKNEMSVVARQVESKISNSINSVSLHIRRGDYVLDNKTNSYHGICDLDYYKKAVEYFKNKLGELNIFVFSDDIAWVKENLRLENLYFVSSPDIKDYEELILMSRCKHNIIANSSFSWWGAWLNANKNKTVITPKKWFQKYNINQKHIVPKSWIRL

SEQ ID NO: 39 - protein, 284 aa, Candidatus magasanikbacteria
MIIVKLSAGLGNQMFQYALGRRLSLDWGDELLFDSSWFYSTREGETPRELELHKFHLLLPEAHPADIEKAKPCTFIKIVKKVKARLDKNVFYRFNKTLLKKRKYVYLDGYFQSYKYFESIRETLLTDFILTDGYSDDAYTTKQEIERVGESVSIHIRRGDFASTCKTWNGLCSIDYYQKALATIQKTHPLVTVFIFSDDIKWAKENLYFDAVPMVFVSRPSLSATEELSLMSLCKHQIIANSTFSWWAAWLNKNVEKIVVAPSRWLVVSNIDTVDLLPPNWIQI

SEQ ID NO: 40 - protein, 305 aa, Diaminobutyricimonas aerilata
MNKSVTQHTEINIGNGVCAYVAGGLGNQLFILAAAWEQAKRLECPLYLDTSNHDVRGGWRFELEDIGAPGVVLGPHSPWTSVRLSKERVLPFPRFTRPVRRRVFFERDSSRYDPAINDIRPGTTIFGYFQSARYFERSADSMHQLIESAPTSPAEADLLAHYAATPRLTLHLRRGDYLNAPESRRVIATTAYARRAYRLLQQMGAAAPLRLFSDSPNLVQEELGDLDLDIEFADPEGVLSPVNTMRAMSYGSGLVMSNSSFSWWAGWLMRRRDSNLPVIAPRPWNETGTAKADLLFPEWIGLDAR

SEQ ID NO: 41 - protein, 273 aa, Flavobacterium magnum
MIVVKLIGGLGNQMFQYAAAKAIAIERNQPLELDVSAFDHYKLHQFALHHFNFSAKVFRPRHILWDKVRSLFIKILHYKERDFGFNARIFDLKADDLYLDGYFQSEKYFLKHSSTIRREFEIISSLKPQTLKVLEEIRNTNSVSLHIRRGDYVGNPLHGTDNELFYRKALGFIETKIENPKLFVFSDDTLWAKQNFKSKHEVIFIDFNDALSNFEDIKLMSCCKHNIIANSSFSWWGAWLNDNPGKLVVAPQQWFNDKSINTSDLIPENWIRF

SEQ ID NO: 42 - protein, 283 aa, Litoreibacter ponti
MIIARIFGGLGNQLFQYATARALAKRACAPLWLDIRLAPPGDHWEFALNHFAIEAKIAQPGELPPDKNSKLRYAAWRYLGGSPKFTRERGLGFNENILSLRGDVYLHGYFQSERYFEDFPGLRQELTIKTPPSDENRRWADDIRAVPSVSLHLRRGDYLTAKGAGSHASCDAAYYERALNHVAEKMDAEPKVFVFSDDPDWARDNLKLPFEMRVAGHNGSDKHYEDMRLISICRHNIIANSTFSWWGAWLNDNPDKVVVAPKVWFANPKLANPDITPESWIRL

SEQ ID NO: 43 - protein, 290 aa, Dysgonomonas alginatilytica
MITLIISGGLGNQMFEYAAGRALSLRHRTNLSIDLYILNKKTKATIRNYELIVFNIETPITSSIFNKIAVKGFGLLKSSNTRRILLSNTGIFRDEKAQCYDSRFKKLSEKMTLFGYFQNENYFRDISEQLRTDFTFQAPLIGRNDEIRSLIELNTSVSIHIRRGDYSNTNSNLPILDISHYKKAIEYISSQISNPYFFIFSDDIDWVKNNLDLSDSNHQFIDWNKNKNSYIDMQLMSLCKHNIIANSSFSWWGAWLNTNPDKLVIAPDKWYKGDNGIYPDGFLPKEWIVL

SEQ ID NO: 44 - protein, 285 aa, Campylobacter hyointestinalis lawsonii
MVIVKILGGLGNQMFQYAYAKSLEQNGYIVKIDISGFKDYVLQKYGLDKYNIDLQISQKHENDKIYKNTLFYKILRKISVNLSSSIKEKDLRFNNKLLNIKDNSYIYGYFQSEKYFKDIREIILKQFTINQEFSDYAKDIEKKILNSQNSCSIHIRRGDYVTNNNILIHGMCSIEYYKKSMKYLENKIANISYFIFSDDIEWVKNNLPIQNGIYIDSKEKRIPHEDIYLMSLCNHNIIANSTFSWWGGWLNQNKNKIVIAPKIWFADKKLQEQSKDIVCQEWIKI

SEQ ID NO: 45 - protein, 290 aa, Phycisphaerales bacterium
MIIVELKGGLGNQMFQYAFARNLAHIHNTTLKTDISGYACSSGMPYALSPFNVQENFATPDEIQSFTTPKQTAAGKWIHSLLHNHPKKAKSYIRVKYPHFNPKYLKLPDNVYLSGYFQSEKYFINIADIIRNEFKIKTEMDSRNGNIAETIQNSQSVSIHIRRGDYITNPKAIQTHGVCGLDYYNRCVEQISLKIKSPRFFVFSDDFDWSRKNIKLPNDAIYIGHNGPNEAYKDLQLMSFCKYHITANSTFSWWGAWLAAYKDKIVFVPKKWFAKNMNTEGMMPDNWIKV

SEQ ID NO: 46 - protein, 261 aa, Akkermansia sp.
MNMERKTGLMNKKYVSPCFLPGMRLGNIMFTLAAACAHARTVGVECRVPWAYNDASLMLRSRLGGWVLPSTPCGTNEPPSWQEPSFAYCPVPSRIRTGGLRGYFQSARYFGGQEAFIRALFAPLTAEKEPGAVGIHIRLGDYRRLRDKHRILDPGFLRRAAGHLSSGKNRLVLFSDEPDEAAEMLARVPAFGRFALEIDRGAPCESLRRMTAMEELVMSCSSFSWWGAWLGNTRKVIVPRDWFVGGVEDYRDIYLPHWVTL

SEQ ID NO: 47 - protein, 304 aa, Helicobacter sp.
MFGYAFAKALEIQYNTEVKLDILFYTKKINRKNNNIRNFELSHFDVHIKYAFGVANRLDKIITSLIPKRFRADFTPSPFNLIKDNKDLHIKIKERYPFPKDTYFEGYFQNLVYFDHIRNILLHDFCLKTPLDLKNKQLQDYILHTHNSVFLHIRRGDYLEYEHSGFINLTYTNYYNTALKTIQEKLGKAHIFIFSNDMVWCKEHFLNGLDSKILQDLTFQFADNNNEGSAAFEMELMRSCKHGIIANSTFSWWAAYLIQYPNKIIVAPNEFFTIVQDSYSVFHDYEDKILPKNWHRIPLTKENK

SEQ ID NO: 48 - protein, 297 aa, Pedobacter sp.
MVIVKLMGGLGNQMFQYAAARSLSFKRKVYLDKSFLDEHKVSSTDFTARDYELDKFKNIQARNGIKNCITFFTDFRFPFNILRYFFARYAILIKQVENEFIDLASCKNYTLIYLDGYFQSEKYFSHIRKQLLKEFEFPQLDTENTAIRNKIIRSENTVSLHIRRGDYNSSEIIHNIHGVLPLTYYHQAITALTLKYGKLSIYIFSDDMKWAKEHLLINDQEVHYVDLNHKKKHWQDMALMSACKHHIIANSSFSWWGAWLADTGGDTFAPLKWFNSDATKFEINDFIPQNWTIVNYG

SEQ ID NO: 49 - protein, 285 aa, Empedobacter brevis
MIAVEIIGGLGNQMFQYATAKAMALHRRDDLLIDKRPFESYSLHSFSLPHFNVTAPYLKEDIVLDLKSTSDKLKAFIKGQKNYTLYQENGLGYDASLFDLQAKNVYLKGYFQSEKYFIKYEDEIRNDFEIVTPLQEKTVEMLTIINEVNAVSLHIRRGDYVTNPEANSVHGTCNLDYYQRAISHIREVVENPIFFIFSDDIQWAKENLTIPETTHFIDFTDASTNYEDIKLMSTCKHNIIANSSFSWWGAWLNKNKSKIVIAPSKWFNVDYHNADDIIPESWMKI

SEQ ID NO: 50 - protein, 261 aa, Akkermansia muciniphila
MNMERKTGLMNKKYVSPCFLPGMRLGNIMFTLAAACAHARTVGVECRVPWAYNDASLMLRSRLGGWVLPSTPCGTNEPPSWQEPSFAYCPVPFRIRTGGLRGYFQSARYFEGQEAFIRALFAPLTAEKEPGAVGIHIRLGDYRLLRDKHRILDPGFLRRAAGHLSSGKNRLVLFSDEPDEAAEMLACVPAFGRFALEIDRGAPCESLRRMTAMEELVMSCSSFSWWGAWLGNTRKVIVPRDWFVGGVEDYRDIYLPHWVTL

SEQ ID NO: 51 - protein, 261 aa, Akkermansia muciniphila
MNMERKTGLMNKKYVSSCFLPGMRLGNIMFTLAAACAHARTVGVECRVPWAYNDASLMLRSRLGGWVLPSTPCGTNEPPSWQEPSFSYCPVPSRIRTGGLRGYFQSARYFEGQEAFIRALFAPLTAEKEPGAVGIHIRLGDYRRLRDKHRILDPGFLRRAAGHLSSGKNRLVLFSDEPDEAAEMLARVPAFGRFALEIDRGAPCESLRRMTAMEELVMSCSSFSWWGAWLGNTRKVIVPHDWFVGGVEDYRDIYLPHWVTL

SEQ ID NO: 52 - protein, 261 aa, Akkermansia muciniphila
MNMERKTGLMNKKYVSPCFLPGMRLGNIMFTLAAACAHARTVGVECRVSWAYNDASLMLRSRLGGWVLPSTPCGTNEPPSWQEPSFSYCPVPSRIRTGGLRGYFQSARYFEGQEAFIRALFAPLTAEKEPGAVGIHIRLGDYRRLRDKHRILDPGFLRRAAGHLSSGKNRLVLFSDEPDEAAEMLARVPAFGRFALEIDRGAPCESLRRMTAMEELVMSCSSFSWWGAWLGNTRKVIVPRDWFVGGVEDYRDIYLPHWVTL

SEQ ID NO: 53 - protein, 319 aa, Chitinophagaceae bacterium
MEKLFRIQAAHRLSGVRAHLIRRHLFQKLISRRDSMIIVQLKGGLGNQMFQYAAARALAARHRTQVLLDTAHYRADQLRNFDLFHLQVAASTAAPEDCAPLKAQSTFSRLAARLTPYSRKRFYKQPFYHFDPHFFELGPDVYLQGYFQSERYFAPLAPQLREEFRFRDAFPEHVRNAAGEMRAGPSVSLHIRRGDYRNPETLRVHGILPFAYYEAAIRKLQERIGPPAFYVFTDDPQWVAANLDIPGARIVSGATSSTHFEDLYLMSHCRHHILANSSFSWWGAWLNTDPGKIVIAPRQWFNEGPVDLQDLMPAGWERI

SEQ ID NO: 54 - protein, 284 aa, Chitinophagaceae bacterium
MIAIQLKGGLGNQLFQYAAARTLAIRQHTEVLLDTSHYREDQLRDFDLFHLNVKARIATASQIDEIRPTGTVGRALSHIAPYRFKRIYQQPFYHYDAGFVRLRGPVYLKGYFQSEAYFRPVAAEIRRELTFRELPLPQIQALGARLRQEQSVSLHIRRGDYKNPETLRVHGILPLEYYRAAVKRITDSRPDARFYVFTDDLEWVRQHLAMPEAMLVSGEITKTHFEDLYLMQHCRNHILANSSFSWWGAWLNPDKEKIVVAPKAWFNEGPKDTQDLIPRSWIRL

SEQ ID NO: 55 - protein, 245 aa, Proteobacteria bacterium
MDKFSVLTSQASPAEVEKRFPLLSFRSLIKIYRRLLRILPKLSRKYYFEKSMAFDHSLFETNPECFIDGYFQSYKYFEGVNDIILQEFQPKEAPDEVNHHLIRVMQVANSVSLHIRRGDYISSAEANKVHGICGLEYYRAAVENLQSRLVDPQYFVFSDDIAWAKENLDLGAGATFVQHNAGSKSYWDLVLMSHCKHNIIANSTFSWWGAWLNQNPDKIVYAPKTWFADSSKNTSDLIPQSWMRI

SEQ ID NO: 56 - protein, 339 aa, Flaviaesturariibacter sp.
MSWHLRVVLTIASANGCNGRCRNGCPTRSLCCISARRRSLRHHSETFISKAPGRFMIVVELKGGLGNQLFQYAAARALAAKHGTGVAFDATHYHEDQLRNFELLHMKVAARKATSGEVAALKPHSFFDRLRNRLAPYERKRFYKQPFFHYDKQFFRLPAKVYLRGYFQSARYFEPVAEIIRQEFRFQQPMPPAVEELGRRLRSEESVSIHIRRGDYKNPETLRVHGIQPLAYYEAAVQLLRERINAPLFYIFTDDPGWVRKNLSIEGAQLVSGPVSATHFEDLYLMQQCRHNIIANSSFSWWGAWLNDNAQKMVIAPNTWFNEGPKDTQNLIPGTWLRL

SEQ ID NO: 57 - protein, 287 aa, Pedobacter sp.
MIVIVKLMGGLGNQMFQYAAARSLSSRKIYFDDSFLNKNNISSEDFTARRFELHIFRRLKVKILNPYAQRLLLTKNKKYDFLKLLLPNSLKRICHIDDSNINENLSKKHQNEILYIDGYFQNPTYFNEIRKSLLNEFAFPELDDKFTGLLNDIENSNSVAIHIRRGDYLKSKINEHHGVLPLSYYKNAVKILEGKLIDPKYFIFSDDPNWCKNNFAFLANKEIVSKENEPWIDMYLISKCKNQIIANSSFSWWGAWLNQFINKIVIAPKNWFNSIESNIVPKEWISL

SEQ ID NO: 58 - protein, 246 aa, Helicobacter jaachi
MYALLRYGILKGNFAFKPDIFALYKYRYDEHLSHKDDRVWQAFTQSAKGSFHPHALPIGYFQNLTYLQGLDSILHKEFCLKTPLTAQNQALKAYIHSLPQSAFLHIRLGDYLEGGTFVRLGSAYYNQAVQILKKHLGKAYIFVFSNNIPWCERNAHKYIDFSGLKVEFVKHNDEGNAAQELELMRACKHGIMANSTFSWWAAYLNDNPHKIVCMPKYFFNDITRIPQSQMIAKQNYILLDHTWAEI

SEQ ID NO: 59 - protein, 295 aa, Helicobacter japonicus
MGGLGNQMFQYAFAKSLEHHLQIPILLDKSWYDNPKNAKMLSLDIFHMDLVYATKTQVIEALNHPNIEQLTESRAFERKPKILRSILKWLGYKKPTFSTPPFEYRAEYLKSNNITYFHGYFQNPRYYQGIDDYIKESLSPPPIKSLENLSKLDQILNTKNSIFIHIRRGDYLSLSWQIDIEYYKKAVATIVNKIENPHFFLFCADREFAHNLDLGYPFIDMTSENISLDNHFEDLILMAHCQHGIVANSSYSWWAAYLIENPHKIIIAPTPWLHSNDEIICDDWIKIQAEKEVRL

SEQ ID NO: 60 - protein, 268 aa, Klebsiella pneumoniae
MLFQVCLAMHLRDMGHVVKIDTLGLENKFKDKVMFFLFKCGITLEECTKAERFICCHAVSTNRIKSKELKIIKLIFPKKLYLEQQWGEIPDDSYNYYFGYFQNINLAKKYCDFFQKGLDLIAEENSFVQPLSNDCFIHIRRGDYCTPSALALHGLMGEEYYNGAVRYFSEDMHFDVFSNDVSWVKSNLSISNIKVMEGEGFKYPDIEDLYKMSRHKYGIIANSTFSFWAALIGSSNINKEIVCPEKWFANEELQKLSYKIKNSDWVYK

SEQ ID NO: 61 - protein, 293 aa, Dysgonomonas capnocytophagoides
MIRVILSGGLGNQMFQYAAGRALSLKLQSELSVDTFLLRKKTKTTVRNFELKVFNIKVRENNHLKNKLITKSFFFLNKYGLKKLIFGLFGIFRDQKAQNFDQRFNNIDRNITLFGYFQNENYFKAISANLREDFRFISPLLGENADIANKIGSTASVSIHIRRGDYLNPNVNLSLLNIEYYQRAISYIQEKITDPVFYIFSDDINWVKKNLDLSQSPHIFIDWNTGNRSFLDMQLMSLCQHNIIANSSFSWWGAWLNCNPDKIVIAPGTWYKNETAADYPIGFIPEKWVILET

SEQ ID NO: 62 - protein, 204 aa, Flavobacterium sp.
MQNDNESCEMVLPLFGNWKFYYGYFQSEQYFKEIADNIRDYFRIKSIYTKAFWQKYGYLFSQKVLAIHIRLGDYLTWGNEAMGGENMTLPDAYFQNALGMIPNIQDYTVLVITDDIENASHKMQWLQGKKIISDTEIMDFQLLMHADKLIISNSSFAWWAAYLNVKNAETFAPEYWLGFKVNTEIPNNVIPSRFKRVAVATCYA

SEQ ID NO: 63 - protein, 291 aa, Pedobacter sp.
MITIKLQGGLGNQMFQYAAARSLSKKIFLDLDFLEVNCTDKLHFTARTFQLGIFENLKIKRSSTNNLLRQNNIAFRVLGKLFKSRIIHIKQTTNDFINLPGSGDLKYIYLDGYFQSEKYFSHIKKEIRDEFKFPALDIKNLDIANQIRNSENATSIHIRRGDYVKSSIVNDIHGTLPKIYYQKAVSKLLLDFPKISIFVFSDDIKWARKHLNFENCNFISNNIKEESWKDMALMSLCKHHIIANSSFSWWGAWLSIYEGQKYAPVNWFNPKKIKYNLNDIIPREWNIIDYD

SEQ ID NO: 64 - protein, 291 aa, Hoeflea phototrophica
MTVRRVISRMHGGLGNQLFQYAVGRAVALRTGSELLLDTREFTSSNPFQYDLGHFSIQAKVANSSELPPGKNRPLAYAWWRKFGRSPRFVREQDLGYNARIETIEADCYLHGYFQSQKYFEDIASILWKDLSFRQAISGENASMAERIQSAPSVSMHIRRGDYLTSAKARSTHGAPDLGYYGRALGEIRARSGSDPVVYLFSDDPDWVRNNMRMDANLVTVAINDGKTAFEDLRLMSLCDHNIIVNSTFSWWGAWLNPSLDKIVVAPKRWFADPKLSNPDITPPGWLRLGD

SEQ ID NO: 65 - protein, 305 aa, Subdoligranulum variabile
MIYAELAGGLGNQMFIYAFARALGLRCGEAVTLLDRQDWRDGAPAHTACALEGLNLVPEVKILAEPGFAKRHLPRQNTAKALMIKYEQRQGLMARDWHDWERRCAPVLNLLGLHFATDGYTPVRRGPARDFLAWGYFQSEAYFADFAPTIRAELRAKQAPAGVWAEKIRAAACPVALHLRRGDYCRPENEILQVCSPAYYARAAAAAAAAYPEATLFVFSDDIDWAKEHLDTAGLPAVWMPRGDAVGDLNLMALCRGFILSNSTYSWWAQYLAGEGRTVWAPDRWFAHTKQTALYQPGWHLIETR

SEQ ID NO: 66 - protein, 288 aa, Crassostrea gigas
MYESKKLDGIACVKFQGRLGNLMMEYVFLYVIAKMKHLYPIVPENSELFQIFDLEKTTLSAINKSTDSCSKLPEYKESWGLSYDEKLLAVPPNKSVRFNGYFQSWKYWIKYEDKIRKLLRFKNPIRQKAHDQMRTIMNKMKFEVNKDSAIVSIHIRRGDYATEGHYKYGKLTPNETYYANALQYFKTRHKNILFVVGSNDIDWSKKALAKEKNVYYSTGNSPAEDIALLSLANHTIMSVGTFGWWIGWMAQGTSIFYKNIFKSQSDFAKEFRNNSTDDFIYPGWIPME

SEQ ID NO: 67 - protein, 265 aa, Crassostrea gigas
MEYVFLYVIAKMKHLYPVVPENSELFQIFNIEKTTLTAIGKSAYSCAKLPVYKERWGLSYDEKLHAIPPYKGVQFDGYFQSWKYWIKYENEIRKLLRFKNPITQKAFTQMRDIIDKMKFEVNKESVIVSIHIRRGDYATEGHYKYGKLTPNETYYANAMQYFKTRHKNILFVVGSNDIDWSKKALAREKNVYYSTGNSPAEDIALLSLANHTIMSVGTFGWWIGWMAQGTSVFYKNIFRPQSDFAKEFRNNSIDDFIYPGWIPME

SEQ ID NO: 68 - protein, 277 aa, uncultured bacterium
MLTLKLKGGLGNQMFQYAASHNLAKNKKTKINFDLSFFSDIEVRDIKRDYLLDKFNISADISFDQKNSISGFRKFLVKVISKFFGEVFYYRLKFLSSKYLDGYFQSEKYFKNVEEDIRKDFTLKDEMGVEAKKIEQQIVNSKNSVSLHIRRGDYVDDLKTNIYHGVCNLDYYKRSIKYLKENFGEINIFVFSDDIAWVKENLAFENLQFVSRPDIKDYEELMLMSKCEHNIIANSSFSWWGAWLNENKNKIIIAPKEWFQKFNINEKHIVPKSWIRL

SEQ ID NO: 69 - protein, 293 aa, Clostridium sp.
MIMLQMTGGMGNQMFTYALYRSLRQKGKEVCIEDFTHYDTPEKNCLQTVFHLDYRKADREVYQRLTDSEPDFLHKVKRKLTGRKEKIYQEKDAIIFEPEVFQTDDVYMIGYFQSGRYFEKAVFDLRKDFTFAWNTFPEKAKKLREQMQAESSVSLHIRRGDYMNGKFASIYGNICTDAYYEAARRYMKEHFGDCRFYLFTDDAEWGRQQESEDTVYVDASEGAGAYVDMALMSCCRHHIIANSSFSWWGAWLDENPDKTVIAPAKWLNISEGKDIYAGLCNCLIDANGSVQGE

SEQ ID NO: 70 - protein, 351 aa, Helobdella robusta
MSFIIKYVYVVALATILIFLITVYFAENFKVGRNFLDSSPVTPPGSKYASLDACTFNSRRTGNLMFALAGLLFVAQNTRRNPILPTNIPYGWMDDYFNTSSIQRLPNEFVYDANKTVVLKERFGPFVYDPIFENPHKNSTVESKEIILLCGYFQNYKYVEKIDKKLKMIFTFYNETQSKVQNFIDHHKKTSAKNRSNVATVGIHIRRGDFLIKFHRNRGFAVVDENFINSTVNYFYKLWTAKNVEFILYFIASEDEAWVNSVVKKLNFARALNKAAFVFSTSNGGPFDMCLISMCDGVITSSGSFSWWAGWLANTTTIYFTGYPRKGSALGEGFDRKTYQKPDWIGFPPEI

SEQ ID NO: 71 - protein, 299 aa, Helicobacter pylori
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 72 - protein, 5 aa, Artificial sequence
  domain 1
  VARIANT (3)..(3): Xaa can be Phe or Tyr
  VARIANT (5)..(5): Xaa can be Asn or Ser
GYXQX

SEQ ID NO: 73 - protein, 7 aa, Artificial sequence
  domain 2
  UNSURE (1)..(1): Xaa can be any distinct amino acid excluding a valine residue
  VARIANT (3)..(3): Xaa can be Leu or Ile
  VARIANT (5)..(5): Xaa can be Arg or Leu
XHXRXGD

SEQ ID NO: 74 - protein, 14 aa, Artificial sequence
  new domain 2
  UNSURE (1)..(1): Xaa can be any naturally occcurring amino acid except Lys and Val
  UNSURE (2)..(5): Xaa can be any naturally occcurring amino acid
  VARIANT (6)..(6): Xaa can be Ile or Leu
  VARIANT (8)..(8): Xaa can be Ile or Leu
  VARIANT (10)..(10): Xaa can be Arg or Leu
  VARIANT (13)..(13): Xaa can be Phe or Tyr
  UNSURE (14)..(14): Xaa can be any naturally occcurring amino acid except Cys and Met
XXXXXXHXRXGDXX

SEQ ID NO: 75 - protein, 239 aa, Akkermansia sp. KLE1798
MRLGNIMFTLAAACSHARTVGVECQVPWTYNDASLMLRSWLGGWVLPSTPCGTNEPPSWQEPSFSYCPIPSNIREGGLCGYFQSVRYFEEQEAFIRALFAPFIAEKESGTLGVHIRLGDYKRLRDKHRVLDLDFLRRAAGHLSPGVRHLVLFSDEPDEAADMLARVPEFGRFTLEIDRGAPCESLHRMTAMEELVISCSSFSWWGAWLGNTRKVIVPHDWFVGGMEDYRDIYLPHWIKL

SEQ ID NO: 76 - protein, 252 aa, Akkermansia muciniphila
MDRLHVSPCFLPGTRLGNVMFTLAAACAHALRVGVPCHVPWNYNDATLLLRSRLGDWVLPSTPCGVNEPAAWQEPSFAYHPIPLHITRGGVCGYFQSARYFEGQEPFIRSLFAPFIAEKEPRTAGIHIRLGDYKQLRHKHRVHGIDFLQRAFTHISPDVNRLILFSDEPEAAADMLVHLPEFRRLPLEIDRNNTCEALRRMTAMEELIISCSSFSWWGAWLGQTRKVIVPRHWFSGVIEDYQDVYLPHWVKL

SEQ ID NO: 77 - protein, 289 aa, Capnocytophaga gingivalis ATCC 33624
MKQKHYIYQQGGLGNQLYILAYAFYLKEKGVGPLCMYSADRQKKDTKDKKKRNVYSQITEKLGFPISGLSYFFFSMAKKMAKKSFFKNYIALQVEKEGQFAVFQEPATQYESKIYLHMGWYQSYLYQTPEFIERLYKIIEELAGNTRQFSITENDVVLHIRRGDFLKEKNRALFHIIETPHYLEGLAKLSQGINLEKVYILSDDFEGIEDIITTLSKQYEVVLVKGNTVLQDLYLLTQFKNYVIGNSTFSWWGAYLSKYKERANIIVPKEPWKIPMPEKSPYPPYWVQI

SEQ ID NO: 78 - protein, 340 aa, Helicobacter sp. CLO-3
MTIINIRDGLGNQMFQYAFAKVLESKGESVVLDTSWYSEEPDSVSKNIANKKNIRNLEITRFDIKIVPYMEIETMLQTQDKFYHFFSFIHKLIAYAPKECLRVGLKSFLPKTRRFHPYKYHIKEVYDSRDLSALPHDILKNALIFGFFQKLSYFKHIDSEIRADFTLCTPLSPANEAMKKRILSTPNAAFIHIRRGDYLNVWQVIKLGKAYYASAIKEILTHVKNPKFFIFSNDIEWCKNNFINLIDSSVFAGKEYEFVEQNGEGDAIEELELMRSCKHGIIANSTFSWWAAYLMENPKKTVIAPSKFFLIPPPQEPNHIDDILPEGWIKTDPTWGNVES

SEQ ID NO: 79 - protein, 320 aa, Helicobacter sp. MIT 17-337
MLIVRLDGGLGNQMFQYAFAKKLESKGYEVGIDISWYADKKNTNPTIANFAQNQHATIRNLEISHYNLSLPLLSDFNPYDFFILHDRFYVLRNKINRFLPRPLRMKNYKFFIPDDVKLCKIIQENKPYFPDYAYFSGFYQNLSCIVEPQDISGDFSLKNPLSQANQALKEQIASFQDSVFLHIRRGDYLAFERYIHLTERYYNQALYMMKTKKDSFHVFVFSNDIQWCKEYFIKQLDNTLMQNLTFEFIGNNDEGNAIIDMELMRTCKHGIIANSTFSWWAAYLLKSKDKIIIAPSRFLQDSTIERMKLLYPQEWTLIEV

(19) United States
(12) Patent Application Publication
(10) Pub. No.: US 2023/0348944 A1
(43) Pub. Date: Nov. 2, 2023

(54) LACTOSE CONVERTING ALPHA-1,2-FUCOSYLTRANSFERASE ENZYMES

(71) Applicant: INBIOSE N.V., Zwijnaarde (BE)

(72) Inventors: Joeri Beauprez, Zwijnaarde (BE); Nausicaä Lannoo, Zwijnaarde (BE); Kristof Vandewalle, Zwijnaarde (BE); Annelies Vercauteren, Zwijnaarde (BE)

(73) Assignees: INBIOSE N.V., Zwijnaarde (BE); INBIOSE N.V., Zwijnaarde (BE)

(21) Appl. No.: 17/785,843

(22) PCT Filed: Dec. 16, 2020
(86) PCT No.: PCT/EP2020/086370

§ 371 Date: Jun. 15, 2022

(30) Foreign Application Priority Data
Dec. 17, 2019 (BE) 20195929

(51) Int. Cl. C12P 19/18; C12N 9/10; C12N 1/20; C12N 15/70

(52) U.S. Cl. CPC C12P 19/18; C12Y 204/01069; C12N 9/1051; C12N 1/205; C12N 15/70; C12R 2001/19

(57) ABSTRACT

The present invention relates to methods for producing 2′ fucosyllactose (2′-FL), as well as newly identified fucosyltransferases, more specifically newly identified lactose binding α-1,2-fucosyltransferase polypeptides, and their applications. Furthermore, the present invention provides methods for producing 2-fucosyllactose (2′FL) using the newly identified α-1,2-fucosyltransferases.

The present invention relates to methods for producing 2′ fucosyllactose (2′-FL), as well as newly identified fucosyltransferases, more specifically newly identified lactose binding alpha-1,2-fucosyltransferase polypeptides, and their applications. Furthermore, the present invention provides methods for producing 2-fucosyllactose (2′FL) using the newly identified alpha-1,2-fucosyltransferases.

### BACKGROUND

Human Milk Oligosaccharides are the third largest solid component in human milk after lactose and lipids. More than 200 free oligosaccharide structures have so far been identified from human milk samples. Today, more than 80 HMO-related compounds have been structurally characterized. These HMOs represent a class of complex oligosaccharides that function as prebiotics. Additionally, the structural homology of HMO to epithelial epitopes accounts for protective properties against bacterial pathogens. Within the infant gastrointestinal tract, HMOs selectively nourish the growth of selected bacterial strains and are, thus, priming the development of a unique gut microbiota in breast milk-fed infants.

Three major HMO categories are present in breast milk: fucosylated neutral HMOs, non-fucosylated neutral HMOs and sialylated acidic HMOs. 2′fucosyllactose (2′FL) is part of the fucosylated group of HMOs. In women who are “secretors” 2′FL is by far the most abundant HMO and constitutes nearly 30% of all HMOs.

Production of fucosylated oligosaccharides requires the action of a fucosyltransferase. Such fucosyltransferases, which belong to the enzyme family of glycosyltransferases, are widely expressed in vertebrates, invertebrates, plants, fungi, yeasts and bacteria. They catalyze the transfer of a fucose residue from a donor, generally guanosine-diphosphate fucose (GDP-fucose) to an acceptor, which include disaccharides, oligosaccharides, (glyco)proteins and (glyco)lipids. The thus fucosylated acceptor substrates are involved in a variety of biological and pathological processes.

Several fucosyltransferases have been identified, e.g. in the bacteria Helicobacter pylori, Escherichia coli, Salmonella enterica, Yersinia, Enterococcus, Shigella, Klebsiella, Bacteroides, in mammals, Drosophila, Caenorhabditis elegans and Schistosoma mansoni, as well as in plants. Fucosyltransferases are classified based on the site of fucose addition into for example alpha-1,2, alpha-1,3, alpha-1,4 and O-fucosyltransferases.

Several alpha-1,2-fucosyltransferases (2′-FTs) are already described. First reports describe the usage of HpFucT2 (Lee et al., 2012, Microb Cell Fact 11:48; Chin et al., 2016, Biotechnol Bioeng. 113:11, 2443-52) or HpFutC (Baumgartner et al., 2013, Microb Cell Fact 12:40; Chin et al., 2015, J. Biotechnol 210, 107-115; Wang et al., 1999, Microbiology 145, 3245-3253) from H. pylori to convert lactose into 2′FL in E. coli strains modified for GDP-fucose synthesis. Tests with WcfB originating from Bacteroides fragilis demonstrated improved 2′FL production in engineered E. coli hosts when compared to HpFucT2 (Chin et al., 2017, J Biotechnol 257, 192-198). Other 2FTs such as WbsJ from E. coli 0128, WbgL from E. coli O126, WbnK and WbwK from E. coli 086, WbiQ from E. coli 0127, FutL from H. mustelae, FutG from C. jejuni, FutN from B. vulgatus ATCC 8482, WcfW from B. fragilis, and Te2FT from Thermosynechococcus elongatus (Te2FT) were also reported to produce 2′FL in engineered E. coli hosts (Chin et al., 2017, J Biotechnol 257, 192-198; Huang et al., 2017, Metab Eng 41, 23-38; Seydametova et al., 2019, Microbiol Res 222, 35-42; US20170081353; US20140024820). Some 2FTs are also reported to transfer GDP-fucose onto lactose resulting in fucosyllactose synthesis in other engineered cells like e.g. Corynebacterium glutamicum (WO2017188684) or Saccharomyces cerevisiae (Liu et al., 2018, ACS Synth Biol 7:11, 2529-2536; Yu et al., 2018, Microb Cell Fact 17, 101).

However, in general, alpha-1,2-fucosyltransferases, also known as 2-fucosyltransferases or 2-fucosyltransferase enzymes, which are needed to produce 2′fucosyllactose, are known to have low affinity for lactose. The low affinity has a negative effect on the productivity of 2′fucosyllactose. In order to improve conversion rates and productivity, there is need for transferases with higher lactose affinity. Furthermore, some alpha-1,2-fucosyltransferases like HpFutC from H. pylori are known to produce difucosyllactose (diFL) as undesired by-product during 2′FL synthesis with a high difucosyllactose concentration to 2′fucosyllactose concentration ratio of 1:5.

Thus, it is an object of the present invention to provide for tools and methods by means of which 2′fucosyllactose can be produced or synthesized in an efficient, time and cost-effective way. Preferably, the invention provides for methods and tools which yields high amounts of the desired product, even more preferably without the undesired by-product diFL or, if diFL is produced, with a lower difucosyllactose concentration to 2′fucosyllactose concentration ratio than 1:5.

### DESCRIPTION

Surprisingly, it has now been found that the newly identified lactose binding alpha-1,2-fucosyltransferase enzymes of the present invention provide for transferases with similar or higher lactose binding and/or transferase properties than the presently known lactose binding alpha-1,2-fucosyltransferase enzymes. In addition, these newly identified transferases do not possess or possess less enzymatic side activity compared to presently known alpha-1,2-fucosyltransferase enzymes that leads to the undesired by-product formation of di-fucosyllactose (diFL) when synthesizing 2′fucosyllactose. When diFL is formed by the newly identified transferases the ratio of concentration of diFL to the concentration of 2′FL is smaller than 1:5, more specifically the diFL concentration to 2′FL concentration ratio is lower than 1:10.

The invention therefore provides methods for producing 2′fucosyllactose (2′FL) using the newly identified lactose binding alpha-1,2-fucosyltransferases. The 2′FL can be obtained by reacting lactose in the presence of alpha-1,2-fucosyltransferase, capable of catalyzing the formation of the 2′fucosyllactose oligosaccharides from lactose and GDP-fucose. Alternatively, it can also be obtained from a microorganism producing an alpha-1,2-fucosyltransferase according to the present invention.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the invention disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims which follow, reference characters used to designate claim steps are provided for convenience of description only, and are not intended to imply any particular order for performing the steps.

According to the present invention, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the present invention. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated. “Mutant” cell or microorganism as used within the context of the present disclosure refers to a cell or microorganism which is genetically engineered or has an altered genetic make-up.

The terms “cell genetically modified for the production of 2′-fucosyllactose” within the context of the present disclosure refers to a cell of a microorganism which is genetically manipulated to comprise at least one of i) a recombinant gene encoding an a 1,2 fucosyltransferase necessary for the synthesis of said 2′-fucosyllactose, ii) a biosynthetic pathway to produce a GDP-fucose suitable to be transferred by said fucosyltransferase to lactose, and/or iii) a biosynthetic pathway to produce lactose or a mechanism of internalization of lactose from the culture medium into the cell where it is fucosylated to produce the 2′-fucosyllactose.

The terms “nucleic acid sequence coding for an enzyme for 2′fucosyllactose synthesis” relates to nucleic acid sequences coding for enzymes necessary in the synthesis pathway to 2′-fucosyllactose, e.g. an enzyme able to transfer the fucose moiety of a GDP-fucose donor substrate onto the 2′ hydroxyl group of the galactose moiety of lactose and thus producing 2′fucosyllactose.

The term “endogenous,” within the context of the present disclosure refers to any polynucleotide, polypeptide or protein sequence which is a natural part of a cell and is occurring at its natural location in the cell chromosome. The term “exogenous” refers to any polynucleotide, polypeptide or protein sequence which originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism. When referring to a gene regulatory sequence or to an auxiliary nucleic acid sequence used for maintaining or manipulating a gene sequence (e.g. a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e. in the genome of a non-genetically engineered organism) is referred to herein as a “heterologous promoter,” even though the promoter may be derived from the same species (or, in some cases, the same organism) as the gene to which it is linked.

The term “polynucleotide encoding a polypeptide” as used herein encompasses polynucleotides that include a sequence encoding a polypeptide of the invention, particularly an alpha-1,2-fucosyltransferase having the amino acid sequence as set forth in SEQ ID Nos. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 75, 76, 77, 78 or 79 of the attached sequence listing. For sake of clarity, also the polynucleotide encoding the polypeptide of SEQ ID NO 71 is a polynucleotide encompassed by the definition, but the polynucleotide of SEQ ID 71 is a prior art alpha-1,2-fucosyltransferase used as a reference. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art. In some embodiments, the present disclosure contemplates making functional variants by modifying the structure of a membrane protein as used in the present invention. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, and in case of the present invention to provide better yield, productivity, and/or growth speed than a cell without the variant.

The term “functional homolog” as used herein describes those molecules that have sequence similarity and also share at least one functional characteristic such as a biochemical activity. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events. Functional homologs are sometimes referred to as orthologs, where “ortholog” refers to a homologous gene or protein that is the functional equivalent of the referenced gene or protein in another species.

Functional homologs can be identified by analysis of nucleotide and polypeptide sequence alignments. For example, performing a query on a database of nucleotide or polypeptide sequences can identify homologs of biomass-modulating polypeptides. Sequence analysis can involve BLAST, Reciprocal BLAST, or PSI-BLAST analysis of non-redundant databases using amino acid sequence of a biomass-modulating polypeptide as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

“Fragment”, with respect to a polynucleotide, refers to a clone or any part of a polynucleotide molecule, particularly a part of a polynucleotide that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, for example at least about 30 nucleotides or at least about 50 nucleotides of any of the sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide.

Fragments may additionally or alternatively include subsequences of polypeptides and protein molecules, or a subsequence of the polypeptide. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example at least about 20 amino acid residues in length, for example at least about 30 amino acid residues in length. Preferentially a fragment is a functional fragment that has at least one property or activity of the polypeptide from which it is derived, such as, for example, the fragment can include a functional domain or conserved domain of a polypeptide. A domain can be characterized, for example, by a Pfam (El-Gebali et al., Nucleic Acids Res. 47 (2019) D427-D432) or Conserved Domain Database (CDD) (https://www.ncbi.nlm.nih.gov/cdd) (Lu et al., Nucleic Acids Res. 48 (2020) D265-D268) designation. The content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art. The PFAM database (https://pfam.xfam.org/) used herein was Pfam version 32.0 released in September 2018.

The terms “alpha-1,2-fucosyltranferase”, “alpha 1,2 fucosyltransferase”, “2-fucosyltransferase, “α-1,2-fucosyltransferase”, “α 1,2 fucosyltransferase”, “2 fucosyltransferase, “2-FT” or “2FT” as used in the present invention, are used interchangeably and refer to a glycosyltransferase that catalyzes the transfer of fucose from the donor substrate GDP-L-fucose, to the acceptor molecule lactose in an alpha-1,2-linkage. A polynucleotide encoding an “alpha-1,2-fucosyltranferase” or any of the above terms, refers to a polynucleotide encoding such glycosyltransferase that catalyzes the transfer of fucose from the donor substrate GDP-L-fucose, to the acceptor molecule lactose in an alpha-1,2-linkage.

The terms “2′ fucosyllactose”, “2′-fucosyllactose”, “alpha-1,2-fucosyllactose”, “alpha 1,2 fucosyllactose”, “α-1,2-fucosyllactose”, “α 1,2 fucosyllactose”, “Galβ-4(Fucα1-2)Glc”, 2FL” or “2′FL” as used in the present invention, are used interchangeably and refer to the product obtained by the catalysis of the alpha-1,2-fucosyltransferase transferring the fucose residue from GDP-L-fucose to lactose in an alpha-1,2-linkage. The terms “difucosyllactose”, “di-fucosyllactose”, “lactodifucotetraose”, “2′,3-difucosyllactose”, “2′,3 difucosyllactose”, “α-2′,3-fucosyllactose”, “α 2′,3 fucosyllactose, “Fucα1-2Galβ 1-4(Fucα1-3)Glc”, “DFLac”, 2′,3 diFL”, “DFL”, “DiFL” or “diFL” as used in the present invention, are used interchangeably. In a preferred embodiment these terms refer to the product obtained by the catalysis of the fucosyltransferase with a preferred alpha-1,2 fucosyltransferase activity, transferring a fucose residue to the 3 carbon of the glucose moiety of a lactose core finally resulting in the formation of an alpha1,3 bound fucose to a lactose or 2′fucosyllactose at the glucose moiety, and finally resulting in a 3-fucosyllactose or 2′,3-difucosyllactose, respectively, or refer to the product obtained by the catalysis of the alpha-1,2-fucosyltransferase transferring the fucose residue to a 3 fucosyllactose resulting in 2′, 3 difucosyllactose. “Oligosaccharide” as the term is used herein and as generally understood in the state of the art, refers to a saccharide polymer containing a small number, typically three to ten, of simple sugars, i.e. monosaccharides.

The term “purified” refers to material that is substantially or essentially free from components which interfere with the activity of the biological molecule. For cells, saccharides, nucleic acids, and polypeptides, the term “purified” refers to material that is substantially or essentially free from components which normally accompany the material as found in its native state. Typically, purified saccharides, oligosaccharides, proteins or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, e.g., 2-fucosyllactose, purity can be determined using methods such as but not limited to thin layer chromatography, gas chromatography, NMR, HPLC, capillary electrophoresis or mass spectroscopy.

The terms “identical” or percent “identity” or % “identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity can be determined using BLAST and PSI-BLAST (Altschul et al., 1990, J Mol Biol 215:3, 403-410; Altschul et al., 1997, Nucleic Acids Res 25: 17, 3389-402). For the purposes of this invention, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM65.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Said control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of said polynucleotide to a polypeptide.

Generally, “operably linked” means that the DNA sequences being linked are contiguous, and, in the case of a secretory leader, contiguous and in reading phase. However, enhancers do not have to be contiguous.

### DETAILED DESCRIPTION OF THE INVENTION

According to a first embodiment, the present invention provides a method for the production of α-1,2-fucosyllactose. The method comprises the steps of:
a) providing a polypeptide with α-1,2-fucosyltransferase activity and with the ability to use lactose as acceptor substrate wherein said polypeptide
   i) comprises an amino acid sequence encoding a conserved domain G-Y-[F/Y]-Q-[N/S] (SEQ ID NO: 72) and an amino acid sequence encoding a conserved domain (X, no K/V)XXXX[I/L]H[I/L]R[R/L]GD[F/Y](X, no C/M) (SEQ ID NO: 74) wherein X can be any distinct amino acid excluding a lysine and a valine residue from the first position of said domain and excluding a cysteine and a methionine residue from the last position of said domain, and/or
   ii) is selected from the group consisting of:
      i) any one of SEQ ID NO 1, 2, 3, 4, 5, 6, 7, 9, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or
      ii) an amino acid sequence having 80% or more sequence identity to the full-length amino acid sequence of any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or
      iii) an amino acid sequence of an allelic variant of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or
      iv) an amino acid sequence of an ortholog of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, and
      v) a functional fragment of an amino acid sequence shown in any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79,
   preferably said polypeptide belongs to the glycosyltransferase 11 (GT11) family
b) contacting the polypeptide with α-1,2-fucosyltransferase activity of step a) with a mixture comprising GDP-fucose as donor substrate, and lactose as acceptor substrate, under conditions where the polypeptide catalyses the transfer of a fucose residue from the donor substrate to the acceptor substrate, thereby producing α-1,2-fucosyllactose.

These newly identified polypeptides comprising both of the above domains and/or selected from the list consisting of SEQ ID NOs 1, 2, 3, 4, 5, 6, 7, 9, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or an amino acid sequence having 80% or more sequence identity to the full-length amino acid sequence of any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or an amino acid sequence of an allelic variant of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or an amino acid sequence of an ortholog of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or a functional fragment of an amino acid sequence shown in any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 provide for an alternative α-1,2-fucosyltransferase having the ability to use lactose as acceptor substrate over the presently known α-1,2-fucosyltransferases. Polypeptides comprising both of the above domains and/or selected from the list consisting of SEQ ID NOs 1, 2, 3, 4, 5, 6, 7, 9, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or an amino acid sequence having 80% or more sequence identity to the full-length amino acid sequence of any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or an amino acid sequence of an allelic variant of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or an amino acid sequence of an ortholog of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or a functional fragment of an amino acid sequence shown in any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 provide for transferases with similar or higher lactose binding and/or similar or higher transferase properties than presently known α-1,2-fucosyltransferases.

A further advantage of using some of the polypeptides newly identified to have the ability to use lactose as acceptor substrate and having α-1,2-fucosyltransferase activity resides in the fact that 2-fucosyllactose is produced with less or no production of the undesired di-fucosyllactose, more specifically with a di-fucosyllactose concentration to 2-fucosyllactose concentration ratio smaller than 1:5,[NL-I1][CS2] preferably smaller than 1:10, more preferably smaller than 1:20, optimally smaller than 1:40.

According to the invention, the method for producing α-1,2-fucosyllactose may be performed in a cell-free system or in a system containing cells. The substrates GDP-fucose and lactose are allowed to react with the alpha-1,2-fucosyltransferase polypeptide for a sufficient time and under sufficient conditions to allow formation of the enzymatic product. These conditions will vary depending upon the amounts and purity of the substrate and enzyme, and whether the system is a cell-free or cellular based system. These variables will be easily adjusted by those skilled in the art.

In cell-free systems, the polypeptide according to the invention, the acceptor substrate(s), donor substrate(s) and, as the case may be, other reaction mixture ingredients, including other glycosyltransferases and accessory enzymes are combined by admixture in an aqueous reaction medium for performing the enzymatic reaction. The enzymes can be utilized free in solution, or they can be bound or immobilized to a support such as a polymer and the substrates may be added to the support. The support may be, e.g., packed in a column.

Cell containing systems or cellular based systems for the synthesis of 2-fucosyllactose as described herein may include genetically modified host cells. According to one aspect of the invention the polypeptide with alpha-1,2-fucosyltransferase activity is produced by a cell producing the polypeptide, e.g. a host cell as described herein. According to another aspect of the invention, the GDP-fucose and/or lactose is provided by a cell producing said GDP-fucose and/or lactose. The cell can be the host cell which is also producing the alpha-1,2-fucosyltransferase. Alternatively, the cell can be another cell than the host cell producing the alpha-1,2-fucosyltransferase, in which case the skilled person would talk about cell coupling. Such cell producing GDP-fucose can express an enzyme converting, e.g., fucose, which is to be added to the host cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus.

In another embodiment, the invention relates to a method for producing α-1,2-fucosyllactose, comprising the following steps:
i) providing a cell genetically modified for the production of α-1,2-fucosyllactose, said cell comprising at least one nucleic acid sequence coding for an enzyme for α-1,2-fucosyllactose synthesis, said cell comprising the expression of a polypeptide with α-1,2-fucosyltransferase activity and with the ability to use lactose as acceptor substrate, wherein said polypeptide a) comprises an amino acid sequence encoding a conserved domain G-Y-[F/Y]-Q-[N/S] (SEQ ID NO: 72) and an amino acid sequence encoding a conserved domain (X, no K/V)XXXX[I/L]H[I/L]R[R/L]GD[F/Y](X, no C/M) (SEQ ID NO: 74) wherein X can be any distinct amino acid excluding a lysine and a valine residue from the first position of said domain and excluding a cysteine and a methionine residue from the last position of said domain, and/or b) is selected from the group consisting of:
   i) any one of SEQ ID NO 1, 2, 3, 4, 5, 6, 7, 9, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or
   ii) an amino acid sequence having 80% or more sequence identity to the full-length amino acid sequence of any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or
   iii) an amino acid sequence of an allelic variant of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or
   iv) an amino acid sequence of an ortholog of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, and
   v) a functional fragment of an amino acid sequence shown in any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79,
preferably said polypeptide belongs to the glycosyltransferase 11 (GT11) family
and
ii) cultivating the cell in a medium under conditions permissive for the production of α-1,2-fucosyllactose.

In a further embodiment, the invention relates to a method for producing alpha-1,2-fucosyllactose, comprising the following steps:
a) providing a host cell expressing a polypeptide with α-1,2-fucosyltransferase activity and with the ability to use lactose as acceptor substrate, wherein said polypeptide i) comprises an amino acid sequence encoding a conserved domain G-Y-[F/Y]-Q-[N/S] (SEQ ID NO: 72) and an amino acid sequence encoding a conserved domain (X, no K/V)XXXX[I/L]H[I/L]R[R/L]GD[F/Y](X, no C/M) (SEQ ID NO: 74) wherein X can be any distinct amino acid excluding a lysine and a valine residue from the first position of said domain and excluding a cysteine and a methionine residue from the last position of said domain, and/or ii) is selected from the group consisting of:
   i) any one of SEQ ID NO 1, 2, 3, 4, 5, 6, 7, 9, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or
   ii) an amino acid sequence having 80% or more sequence identity to the full-length amino acid sequence of any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or
   iii) an amino acid sequence of an allelic variant of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or
   iv) an amino acid sequence of an ortholog of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, and
   v) a functional fragment of an amino acid sequence shown in any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79,
preferably said polypeptide belongs to the glycosyltransferase 11 (GT11) family
b) growing, under suitable nutrient conditions permissive for the production of the α-1,2-fucosyllactose, and permissive for the expression of a polypeptide with α-1,2-fucosyltransferase activity, the host cell of step a);
c) providing simultaneously or subsequently to step b) a donor substrate GDP-fucose and the acceptor substrate lactose, in order for the α-1,2-fucosyltransferase polypeptide to catalyse the transfer of a fucose residue from GDP-fucose to lactose, thereby producing 2-fucosyllactose.

According to yet another embodiment, the production of said 2′-fucosyllactose in the methods as described herein is performed by means of a heterologous or homologous (over)expression of the polynucleotide encoding the alpha-1,2-fucosyltransferase by the cell.

In the methods of the invention as described herein the host cell can be transformed or transfected to express an exogenous polypeptide as described herein and with alpha-1,2-fucosyltransferase activity and with the ability to use lactose as an acceptor substrate. As such, the invention relates to a method for producing alpha-1,2-fucosyllactose using a host cell, comprising the following steps:
a) growing, a host cell transformed or transfected to express an exogenous polypeptide with alpha-1,2-fucosyltransferase activity and with the ability to use lactose as an acceptor substrate, wherein the polypeptide is set forth herein; and
b) providing, simultaneously or subsequently to step a), a donor substrate GDP-fucose and an acceptor substrate lactose, wherein the alpha-1,2-fucosyltransferase polypeptide catalyzes the transfer of a fucose residue from the donor substrate to the acceptor substrate, thereby producing alpha-1,2-fucosyllactose.

Preferably the exogenous polypeptide with alpha-1,2-fucosyltransferase activity and with the ability to use lactose as an acceptor substrate as used herein, produces no difucosyllactose or less difucosyllactose with a diFL concentration to 2′FL concentration ratio smaller than 1:5.

The ratio concentration diFL to concentration 2′FL can be less than 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180; 1:190, 1:200, 1:300, 1:400, 1:500, 1:600, 1:700, 1:800, 1:900, 1:1000.

In a preferred embodiment the ratio diFL concentration on 2′FL concentration of lower than 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180; 1:190, 1:200, 1:300, 1:400, 1:500, 1:600, 1:700, 1:800, 1:900, 1:1000 is obtained within a production process resulting in a final lactose concentration of lower than 25 g/L, 20 g/L, 15 g/L 10 g/L, 9 g/L, 8 g/L, 7 g/L, 6 g/L, 5 g/L, 4 g/L, 3 g/L, 2 g/L, 1 g/L, 0.5 g/L, 0.25 g/L, 0.1 g/L or 0 g/L.

In another embodiment the ratio diFL concentration on 2′FL concentration of lower than 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180; 1:190, 1:200, 1:300, 1:400, 1:500, 1:600, 1:700, 1:800, 1:900, 1:1000 is obtained within a production process wherein the lactose concentration is fed at substrate limiting conditions, wherein the substrate limitation is defined as the concentration in the bioreactor that determines the rate of conversion of the substrate.

In another embodiment the ratio diFL concentration on 2′FL concentration of lower than 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180; 1:190, 1:200, 1:300, 1:400, 1:500, 1:600, 1:700, 1:800, 1:900, 1:1000 is obtained within a production process wherein the lactose is formed in the cell at rate limiting conditions.

In another embodiment, the 2′fucosyllactose purity in the broth is higher than about 80%, such as 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% in broth. As used herein, the 2′-fucosyllactose purity is defined as the ratio of the 2′FL concentration to the sum of the 2′FL concentration, the diFL concentration and the lactose concentration ([2′FL]/([2′FL]+[diFL]+[lactose])).

According to the invention, the GDP-fucose and/or lactose can be fed to the host cell in the fermentation medium or aqueous culture medium. Alternatively, the GDP-fucose and/or lactose can be provided by an enzyme simultaneously expressed in the host cell or by the metabolism of the host cell. Accordingly, the host cell will also produce the alpha-1,2-fucosyltransferase next to the GDP-fucose and/or lactose. In another embodiment, the GDP-fucose and/or lactose can be produced by a cell which is another cell than the host cell producing the alpha-1,2-fucosyltransferase, in which case the skilled person would talk about cell coupling. Such cell producing GDP-fucose can express an enzyme converting, e.g., fucose, which is to be added to the host cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus.

According to yet another embodiment, the production of said alpha-1,2-fucosyllactose is performed by means a host cell as described herein comprising a heterologous or homologous (over)expression of the polynucleotide encoding the alpha-1,2-fucosyltransferase.

In a further aspect, the present invention provides for a method for producing alpha-1,2-fucosyllactose as described herein, wherein the method further comprises a step of separating said alpha-1,2-fucosyllactose from the host cell or the medium of its growth.

The term “separating” means harvesting, collecting or retrieving the alpha-1,2-fucosyllactose from the reaction mixture and/or from the cell producing the alpha-1,2-fucosyltransferase, the host cells, the medium of its growth and/or the reaction mixture as explained herein, the 2-fucosyllactose produced by the use of the newly identified alpha-1,2-fucosyltransferase according to the invention.

In case α-1,2-fucosyllactose is made by use of cells or fermentation, the 2′FL can be separated in a conventional manner from the aqueous culture medium, in which the mixture was made. In case the alpha-1,2-fucosyllactose is still present in the cells producing the alpha-1,2-fucosyllactose, conventional manners to free or to extract the alpha-1,2-fucosyllactose out of the cells can be used, such as cell destruction using high pH, heat shock, sonication, French press, homogenization, enzymatic hydrolysis, chemical hydrolysis, solvent hydrolysis, detergent, hydrolysis, . . . . The culture medium, reaction mixture and/or cell extract, together and separately called 2′FL containing mixture, can then be further used for separating the 2′FL. This preferably involves clarifying the 2′FL containing mixtures to remove suspended particulates and contaminants, particularly cells, cell components, insoluble metabolites and debris produced by culturing the genetically modified cell and/or performing the enzymatic reaction. In this step, the 2′FL containing mixture can be clarified in a conventional manner. Preferably, the 2′FL containing mixture is clarified by centrifugation, flocculation, decantation and/or filtration. A second step of separating the 2′FL from the 2′FL containing mixture preferably involves removing substantially all the proteins, as well as peptides, amino acids, RNA and DNA and any endotoxins and glycolipids that could interfere with the subsequent separation step, from the 2′FL containing mixture, preferably after it has been clarified. In this step, proteins and related impurities can be removed from the 2′FL containing mixture in a conventional manner. Preferably, proteins, salts, by-products, colour and other related impurities are removed from the 2′FL containing mixture by ultrafiltration, nanofiltration, reverse osmosis, microfiltration, activated charcoal or carbon treatment, tangential flow high-performance filtration, tangential flow ultrafiltration, affinity chromatography, ion exchange chromatography (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, while 2′FL remains in the 2′FL containing mixture. 2′FL is further separated from the reaction mixture and/or culture medium and/or cell with or without further purification steps by evaporation, lyophilization, crystallization, precipitation, and/or drying, spray drying.

In an even further aspect, the present invention also provides for a further purification of the alpha-1,2-fucosyllactose. A further purification of said 2-fucosyllactose may be accomplished, for example, by use of (activated) charcoal or carbon, nanofiltration, ultrafiltration or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the product. Another purification step is to dry, spray dry or lyophilize alpha-1,2-fucosyllactose.

The separated and preferably also purified 2′FL can be used as a supplement in infant formulas and for treating various diseases in newborn infants.

Another aspect of the invention provides for a method wherein the polypeptide with α-1,2-fucosyltransferase activity and with the ability to use lactose as acceptor substrate as described herein and preferably also the 2′FL is produced in and/or by a fungal, yeast, bacterial, insect, animal or plant expression system or cell as described herein. The expression system or cell is chosen from the list comprising a bacterium, a yeast, or a fungus, or, refers to a plant or animal cell. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the present invention specifically relates to a mutated and/or transformed Escherichia coli host cell or strain as indicated above wherein said E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Zygosaccharomyces, Pichia, Komagataella, Hansenula, Kluyveromyces, Debaromyces, Yarrowia or Starmerella. The latter fungus belongs preferably to the genus Rhizopus, Dictyostelium, Penicillium, Mucor or Aspergillus.

According to a further aspect of the invention, the polynucleotide encoding the polypeptide with alpha-1,2-fucosyltransferase activity is adapted to the codon usage of the respective cell or expression system. In a further preferred embodiment, the method of the invention uses a culture medium for growth of the host cell or microorganism comprising the alpha-1,2-fucosyltransferase of the invention, wherein the lactose concentration in the culture medium ranges from 50 to 150 g/L. Such lactose concentration in the culture medium can be 50 g/L, 55 g/L, 60 g/L, 65 g/L, 70 g/L, 75 g/L, 80 g/L, 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, or 150 g/L.

In a further preferred embodiment, the method of the invention uses a culture medium for growth of the host cell or microorganism comprising the alpha-1,2-fucosyltransferase of the invention wherein GDP-fucose in the culture medium is present in a concentration enabling the host cell to synthesize 2′-fucosyllactose with a diFL concentration to 2′fucosyllactose concentration ratio of less than 1:5.

In a further preferred embodiment, the method of the invention uses a culture medium for growth of the host cell or microorganism comprising the alpha-1,2-fucosyltransferase of the invention wherein GDP-fucose in the culture medium is present in a concentration enabling the host cell to synthesize 2′-fucosyllactose with a purity of at least 80%.

In a further preferred embodiment, the method of the invention uses a culture medium for growth of the host cell or microorganism comprising the alpha-1,2-fucosyltransferase of the invention wherein a carbon-based substrate is present in a concentration enabling the host cell to synthesize GDP-fucose at an optimal concentration for 2′fucosyllactose synthesis with a diFL concentration to 2′fucosyllactose concentration ratio of less than 1:5.

In a further preferred embodiment, the method of the invention uses a culture medium for growth of the host cell or microorganism comprising the alpha-1,2-fucosyltransferase of the invention wherein a carbon-based substrate is present in a concentration enabling the host cell to synthesize GDP-fucose at an optimal concentration for 2′fucosyllactose synthesis with a purity of at least 80%.

In a further preferred embodiment, the method of the invention produces a final concentration of 2-fucosyllactose ranging between 70 g/L to 200 g/L. Such 2′FL concentration being 70 g/L, 75 g/L, 80 g/L, 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, 150 g/L, 155 g/L, 160 g/L, 165 g/L, 170 g/L, 175 g/L, 180 g/L, 185 g/L, 190 g/L, 195 g/L, or 200 g/L. Higher lactose concentrations in the culture medium can provide even higher 2′FL final concentrations obtained in the production method.

In a further preferred embodiment, the method of the invention produces a final concentration of 2′FL ranging between 70 g/L to 200 g/L as explained above, and wherein the diFL concentration to 2′FL concentration ratio is lower than 1:5, more preferably 1:10 even more preferably 1:40.

Another aspect of the invention provides for a method for an improved rate of fucosylation of lactose by an increased GDP-fucose supply and wherein the diFL concentration to 2′FL concentration ratio is lower than 1:5. An alternative aspect of the invention provides for a method for an improved rate of fucosylation of lactose by an increased GDP-fucose supply and wherein the final 2′FL has a purity of at least 80%. In a preferred embodiment of present invention, the polypeptide of present invention with α-1,2-fucosyltransferase activity having the ability to use lactose as acceptor substrate and having less fucosyltransferase activity on 2′FL compared to HpFutC from H. pylori with SEQ ID NO 71 from present invention has improved affinity for GDP-fucose. [NL-13]

[CS4][NL-15] In the methods of the invention as described herein the polypeptide with α-1,2-fucosyltransferase activity and with the ability to use lactose as acceptor substrate i) comprises an amino acid sequence encoding a conserved domain G-Y-[F/Y]-Q-[N/S] (SEQ ID NO: 72) and an amino acid sequence encoding a conserved domain (X, no K/V)XXXX[I/L]H[I/L]R[R/L]GD[F/Y](X, no C/M) (SEQ ID NO: 74) wherein X can be any distinct amino acid excluding a lysine and a valine residue from the first position of said domain and excluding a cysteine and a methionine residue from the last position of said domain, and/or ii) is selected from the group consisting of:
i) any one of SEQ ID NO 1, 2, 3, 4, 5, 6, 7, 9, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or
ii) an amino acid sequence having 80% or more sequence identity to the full-length amino acid sequence of any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or
iii) an amino acid sequence of an allelic variant of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or
iv) an amino acid sequence of an ortholog of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, and
v) a functional fragment of an amino acid sequence shown in any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79,

preferably said polypeptide belongs to the glycosyltransferase 11 (GT11) family.

Within the scope of the present invention, such polypeptide proved to have lactose binding alpha-1,2-fucosyltransferase activity and preferably has less or no enzymatic side activity on 2-fucosyllactose compared to the presently known alpha-1,2-fucosyltransferase enzymes.

The amino acid sequence used herein can be a sequence chosen from SEQ ID NO 1, 2, 3, 4, 5, 6, 7, 9, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing. The amino acid sequence can also be an amino acid sequence that has greater than about 80% sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length amino acid sequence of any one of SEQ NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79.

Furthermore, within the scope of the present invention, the amino acid sequence can be a variant of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79.

Further included in the scope of the invention is an alpha 1,2-fucosyltransferase polypeptide as described herein which is optionally further modified by an N-terminal and/or C-terminal amino acid stretch. Such amino acid stretch is to be understood as an addition of polypeptide sequences at the N-terminus and/or C-terminus of the polypeptide. For example, polypeptide sequences may be fused to the alpha-1,2-fucosyltransferase polypeptide in order to effectuate additional enzymatic activity. Such amino acid stretch can be a specific tag and/or HQ-tag; an extension of up to 20 amino acids, e.g. 1, 2, 3, 4, 5, 6, 7, 8, 9, 10, 11, 12, 13, 14, 15, 16, 17, 18, 19 or 20 amino acids; such extension can also be 25, 30, 35, 40, 45, 50, 55, 60, 65, 70 or more amino acids long. The optional N-terminal and/or C-terminal amino acid stretch can also be a tag for purification, a tag for increasing the solubility of the polypeptide, a tag or amino acid stretch for metabolon formation, a tag for protein metabolomics, a tag for substrate binding, another polypeptide with the same or a different function in a gene fusion, such as but not limited to a polypeptide coding for GDP-fucose synthase, galactosyltransferase, fucosyltransferase, bifunctional fucose kinase/fucose-1-phosphate guanylyltransferase or fucose-1-phosphate guanylyltransferase, wherein said other polypeptide is optionally fused to the alpha-1,2-fucosyltransferase polypeptide via a peptide linker. For example, the alpha-1,2-fucosyltransferase polypeptide as described herein optionally includes one or more exogenous affinity tags, e.g., purification or substrate binding tags, such as a 6 His tag sequence, a GST tag, a HQ tag, an HA tag sequence, a plurality of 6 His tag sequences, a plurality of GST tags, a plurality of HA tag sequences, a SNAP-tag, a SUMOstar tag. Other examples include proteolytic cleavage sites, retention sites, cleavage sites, polyhistidine tags, biotin, avidin, BiTag sequences, S tags, enterokinase sites, thrombin sites, antibodies or antibody domains, antibody fragments, antigens, receptors, receptor domains, receptor fragments, ligands, dyes, acceptors, quenchers, or combinations thereof.

In addition, alpha-1,2-fucosyltransferase polypeptides may include proteins or polypeptides that represent functionally equivalent polypeptides. Such an equivalent alpha-1,2-fucosyltransferase polypeptide may contain deletions, additions or substitutions of amino acid residues within the amino acid sequence encoded by the alpha-1,2-fucosyltransferase polynucleotides described herein, but which results in a silent change, thus producing a functionally equivalent alpha-1,2-fucosyltransferase. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; planar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Within the context of this invention, “functionally equivalent”, as used herein, refers to a polypeptide capable of exhibiting a substantially similar in vivo activity as the lactose binding alpha-1,2-fucosyltransferase polypeptides of the present invention as judged by any of a number of criteria, including but not limited to enzymatic activity. Included within the scope of the invention are alpha-1,2-fucosyltransferase proteins, polypeptides, and derivatives (including fragments) which are differentially modified during or after translation. Furthermore, non-classical amino acids or chemical amino acid analogues can be introduced as a substitution or addition into the alpha-1,2-fucosyltransferase polypeptide sequence.

The alpha-1,2-fucosyltransferase polypeptide may be produced by expression by polynucleotides produced via recombinant DNA technology using techniques well known in the art. Methods which are well known to those skilled in the art can be used to construct expression vectors containing alpha-1,2-fucosyltransferase coding sequences and appropriate transcriptional and/or translational control signals. These methods include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates). Alternatively, the alpha-1,2-fucosyltransferase polypeptide may be produced by direct synthesis, by extraction of the cell which produces the polypeptide in nature or within a cell free and/or in vitro system. The suitability of the newly identified alpha-1,2-fucosyltransferases having the ability to bind lactose to be used for producing 2-fucosyllactose with less or no difucosyllactose by-product formation and preferably with a difucosyllactose concentration to 2′FL concentration ratio smaller than 1:5, and even more preferably with a 2′FL purity of 80% or more, is highly surprising, and, thus, their use represents an excellent tool to easily, efficiently and cost-effectively produce 2′-fucosyllactose.

The polynucleotide encoding the alpha-1,2-fucosyltransferase polypeptide may be produced via recombinant DNA technology using techniques well known in the art. Methods which are well known to those skilled in the art can be used to construct expression vectors containing alpha-1,2-fucosyltransferase coding sequences and appropriate transcriptional and/or translational control signals. These methods include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to another aspect of the invention, a vector can be provided, containing a polynucleotide encoding said polypeptide with alpha-1,2-fucosyltransferase activity as described herein, wherein the polynucleotide is operably linked to control sequences recognized by a host cell transformed with the vector. In a particularly preferred embodiment, the vector is an expression vector, and, according to another aspect of the invention, the vector can be present in the form of a plasmid, cosmid, phage, liposome, or virus.

Thus, the polynucleotide encoding the polypeptide with alpha-1,2-fucosyltransferase activity as described herein, may, e.g., be comprised in a vector which is to be stably transformed/transfected into host cells. In the vector, the polynucleotide encoding a polypeptide with alpha-1,2-fucosyltransferase activity as described herein is under control of a promoter. The promoter can be e.g. an inducible promoter, so that the expression of the gene/polynucleotide can be specifically targeted, and, if desired, the gene may be overexpressed in that way. The promoter can also be a constitutive promoter.

A great variety of expression systems can be used to produce the polypeptides of the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above.

For recombinant production, host cells can be genetically engineered to incorporate expression systems or portions thereof or polynucleotides of the invention. Introduction of a polynucleotide into the host cell can be effected by methods described in many standard laboratory manuals, such as Davis et al., Basic Methods in Molecular Biology, (1986), and Sambrook et al., 1989, supra.

According to a further aspect, the invention provides a host cell genetically modified for the production of alpha-1,2-fucosyllactose, wherein the host cell comprises at least one nucleic acid sequence coding for an enzyme for 2′-fucosyllactose synthesis and wherein said cell comprises the expression of a polypeptide with α-1,2-fucosyltransferase activity and with the ability to use lactose as acceptor substrate.

Said Polypeptide
i) comprises an amino acid sequence encoding a conserved domain G-Y-[F/Y]-Q-[N/S] (SEQ ID NO: 72) and an amino acid sequence encoding a conserved domain (X, no K/V)XXXX[I/L]H[I/L]R[R/L]GD[F/Y](X, no C/M) (SEQ ID NO: 74) wherein X can be any distinct amino acid excluding a lysine and a valine residue from the first position of said domain and excluding a cysteine and a methionine residue from the last position of said domain, and/or
ii) is selected from the group consisting of:
   i) any one of SEQ ID NO 1, 2, 3, 4, 5, 6, 7, 9, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or
   ii) an amino acid sequence having 80% or more sequence identity to the full-length amino acid sequence of any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or
   iii) an amino acid sequence of an allelic variant of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or
   iv) an amino acid sequence of an ortholog of an amino acid sequence shown in any one of SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, and
   v) a functional fragment of an amino acid sequence shown in any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79,
preferably said polypeptide belongs to the glycosyltransferase 11 (GT11) family.

As used herein, the term “host cell” is presently defined as a cell which has been transformed or transfected or is capable of transformation or transfection by an exogenous polynucleotide sequence, thus containing at least one sequence not naturally occurring in said host cell.

A variety of host-expression vector systems may be utilized to express the alpha-1,2-fucosyltransferase polynucleotides of the invention. Such host-expression systems represent vehicles by which the coding sequences of interest may be produced and subsequently purified, but also represent cells which, when transformed or transfected with the appropriate nucleotide coding sequences, exhibit the alpha-1,2-fucosyltransferase gene product of the invention in situ.

According to another aspect of the invention, a host cell for the production of 2-fucosyllactose is provided wherein the host cell comprises a sequence consisting of a polynucleotide encoding a polypeptide with lactose binding alpha-1,2-fucosyltransferase activity as described herein, wherein the polynucleotide is a sequence foreign to the host cell and wherein the sequence is integrated in the genome of the host cell. The polynucleotide is operably linked to control sequences recognized by the host cell.

According to an alternative aspect of the invention, a host cell for the production of 2-fucosyllactose is provided wherein the host cell contains a vector comprising a polynucleotide encoding a polypeptide with lactose binding alpha-1,2-fucosyltransferase activity described herein, wherein the polynucleotide being operably linked to control sequences recognized by a host cell transformed with the vector.

[NL-16] In a further aspect, the present invention also provides for a method for the production of α-1,2-fucosyllactose, comprising the steps of: a) providing a cell as described herein, and b) cultivating the cell in a medium under conditions permissive for the production of α-1,2-fucosyllactose.

Preferably, said α-1,2-fucosyllactose is separated from the cultivation as described herein. Preferably, also a purification can be done as described herein.

In another further aspect, the invention provides for use of the cell as described herein for the production of 2-fucosyllactose.

According to a further aspect of the invention, a microorganism is provided expressing the alpha-1,2-fucosyltransferase as described herein.

The term micro-organism or organism or cell or host cell as used herein refers to a microorganism chosen from the list comprising a bacterium, a yeast, or a fungus, or, refers to a plant or animal cell. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains —designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the present invention specifically relates to a mutated and/or transformed Escherichia coli host cell or strain as indicated above wherein said E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus such as Bacillus subtilis or B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Zygosaccharomyces, Pichia, Komagataella, Hansenula, Kluyveromyces, Debaromyces, Yarrowia or Starmerella. The latter fungus belongs preferably to the genus Rhizopus, Dictyostelium, Penicillium, Mucor or Aspergillus.

According to another aspect of the invention, the polynucleotide encoding the polypeptide with lactose binding alpha-1,2-fucosyltransferase activity is adapted to the codon usage of the respective host cell.

A further aspect of the invention provides for the use of a polypeptide as described herein for the production of alpha-1,2-fucosyllactose.

A further aspect of the invention provides for the use of a vector as described herein for the production of alpha-1,2-fucosyllactose.

Accordingly, the invention also relates to the 2-fucosyllactose obtained by the methods according to the invention, as well as to the use of a polynucleotide, the vector, host cells, microorganisms or the polypeptide as described above for the production of 2-fucosyllactose. The alpha-1,2-fucosyllactose may be used as food additive, prebiotic, symbiotic, for the supplementation of baby food, adult food or feed, or as either therapeutically or pharmaceutically active compound. With the novel methods, alpha-1,2-fucosyllactose can easily and effectively be provided, without the need for complicated, time and cost consuming synthetic processes.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, enzymatic reactions and purification steps are performed according to the manufacturer's specifications.

Further advantages follow from the specific embodiments, the examples and the attached drawings.

It goes without saying that the abovementioned features and the features which are still to be explained below can be used not only in the respectively specified combinations, but also in other combinations or on their own, without departing from the scope of the present invention.

The present invention relates to the following specific embodiments:
1. A method for the production of α-1,2-fucosyllactose, the method comprising the steps of:
   a) providing a polypeptide with α-1,2-fucosyltransferase activity and with the ability to use lactose as acceptor substrate wherein said polypeptide
      i) comprises an amino acid sequence encoding a conserved domain G-Y-[F/Y]-Q-[N/S] (SEQ ID NO: 72) and an amino acid sequence encoding a conserved domain (X, no K/V)XXXX[I/L]H[I/L]R[R/L]GD[F/Y](X, no C/M) (SEQ ID NO: 74) wherein X can be any distinct amino acid excluding a lysine and a valine residue from the first position of said domain and excluding a cysteine and a methionine residue from the last position of said [NL-17] domain, and/or
      ii) is selected from the group consisting of:
         i) any one of SEQ ID NO 01, 02, 03, 04, 05, 06, 07, 09, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or
         ii) an amino acid sequence having 80% or more sequence identity to the full-length amino acid sequence of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or
         iii) an amino acid sequence of an allelic variant of an amino acid sequence shown in any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or
         iv) an amino acid sequence of an ortholog of an amino acid sequence shown in any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, and
         v) a functional fragment of an amino acid sequence shown in any one of SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79,
   preferably said polypeptide belongs to the glycosyltransferase 11 (GT11) family,
   b) contacting the polypeptide with α-1,2-fucosyltransferase activity of step a) with a mixture comprising GDP-fucose as donor substrate, and lactose as acceptor substrate, under conditions where the polypeptide catalyses the transfer of a fucose residue from the donor substrate to the acceptor substrate, thereby producing α-1,2-fucosyllactose,
   c) optionally separating said α-1,2-fucosyllactose.
2. Method according to embodiment 1 for the production of α-1,2-fucosyllactose resulting in a diFL concentration to 2′fucosyllactose concentration ratio of less than 1:5, preferably a α-1,2-fucosyllactose purity of 80% or more.
3. Method according to any one of embodiment 1 or 2, wherein said polypeptide is provided in a cell free system.
4. Method according to any one of embodiment 1 or 2, wherein said polypeptide is produced by a cell comprising a polynucleotide encoding said polypeptide.
5. Method according to any one of embodiment 1, 2 or 4, wherein said GDP-fucose and/or lactose is provided by a cell producing said GDP-fucose and/or lactose.
6. A method according to any one of embodiments 1, 2, 4 or 5, the method comprising the steps of:
   i) providing a cell genetically modified for the production of α-1,2-fucosyllactose, said cell comprising at least one nucleic acid sequence coding for an enzyme for α-1,2-fucosyllactose synthesis,
   said cell comprising the expression of said polypeptide with α-1,2-fucosyltransferase activity and with the ability to use lactose as acceptor substrate,
   ii) cultivating the cell in a medium under conditions permissive for the production of α-1,2-fucosyllactose,
   iii) optionally separating the α-1,2-fucosyllactose from the cultivation.
7. Method according to embodiment 4, the method comprising the steps of:
   a) providing a host cell expressing said polypeptide with α-1,2-fucosyltransferase activity and with the ability to use lactose as acceptor substrate,
   b) growing, under suitable nutrient conditions permissive for the production of the α-1,2-fucosyllactose, and permissive for the expression of said polypeptide with α-1,2-fucosyltransferase activity, said host cell;
   c) providing simultaneously or subsequently to step b) a donor substrate GDP-fucose and the acceptor substrate lactose, in order for the α-1,2-fucosyltransferase polypeptide to catalyse the transfer of a fucose residue from GDP-fucose to lactose, thereby producing α-1,2-fucosyllactose;
   d) optionally separating said α-1,2-fucosyllactose from the host cell or the medium of its growth.
8. A method according to any one of embodiment 6 or 7, wherein the host cell is transformed or transfected to express an exogenous polypeptide with alpha-1,2-fucosyltransferase activity and with the ability to use lactose as an acceptor substrate.
9. Method according to any one of embodiment 4 to 8 characterized in that the GDP-fucose and/or lactose is provided by an enzyme simultaneously expressed in the host cell or by the metabolism of the host cell[CS8][NL-19].
10. The method of any one of embodiments 1 to 9, further comprising purification of α-1,2-fucosyllactose.
11. Method for the production of 2′-fucosyllactose according to any one of the preceding embodiments, the method further comprising at least one of the following steps:
   i) adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter)[NL10][CS11], preferably in a continuous manner, and preferably so that the final volume of the culture medium is not more than three-fold, preferably not more than two-fold, more preferably less than 2-fold of the volume of the culture medium before the addition of said lactose feed;
   ii) adding to the culture medium a GDP-fucose feed at a concentration enabling the host cell to synthesize 2′-fucosyllactose with a diF[NL-I12]L concentration to 2′fucosyllactose concentration ratio of less than 1:5, preferably in a continuous manner and preferably so that the final volume of the culture medium is not more than three-fold, preferably not more than two-fold, more preferably less than 2-fold of the volume of the culture medium before the addition of said GDP-fucose feed;
   iii) adding to the culture medium a carbon-based substrate feed at a concentration enabling the host cell to synthesize GDP-fucose at an optimal concentration for 2′fucosyllactose synthesis with a diFL concentration to 2′fucosyllactose concentration ratio of less than 1:5, preferably in a continuous manner;
   iv) adding to the culture medium a carbon-based substrate feed at a concentration enabling the host cell to internally synthesize lactose at an optimal concentration for 2′fucosyllactose synthesis;
   v) adding a lactose feed, a GDP-fucose feed and/or a carbon-based substrate feed in a continuous manner to the culture medium over the course of 1 day, 2 days, 3 days, 4 days, 5 days by means of at least one feeding solution;
   vi) adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
   vii) said method resulting in a 2′-fucosyllactose concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
12. Host cell genetically modified for the production of alpha-1,2-fucosyllactose, wherein the host cell comprises at least one nucleic acid sequence coding for an enzyme for α-1,2-fucosyllactose synthesis; said cell comprising the expression of a polypeptide with α-1,2-fucosyltransferase activity and with the ability to use lactose as acceptor substrate wherein said polypeptide
   i) comprises an amino acid sequence encoding a conserved domain G-Y-[F/Y]-Q-[N/S] (SEQ ID NO: 72) and an amino acid sequence encoding a conserved domain (X, no K/V)XXXX[I/L]H[I/L]R[R/L]GD[F/Y](X, no C/M) (SEQ ID NO: 74) wherein X can be any distinct amino acid excluding a lysine and a valine residue from the first position of said domain and excluding a cysteine and a methionine residue from the last position of said domain; and/or,
   ii) is selected from the group consisting of:
      i) any one of SEQ ID NO 01, 02, 03, 04, 05, 06, 07, 09, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or
      ii) an amino acid sequence having 80% or more sequence identity to the full-length amino acid sequence of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or
      iii) an amino acid sequence of an allelic variant of an amino acid sequence shown in any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or
      iv) an amino acid sequence of an ortholog of an amino acid sequence shown in any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, and
      v) a functional fragment of an amino acid sequence shown in any one of SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79,
   preferably said polypeptide belongs to the glycosyl transferase 11 (GT11) family.
13. Cell according to embodiment 12, the host cell comprising
   i) a sequence comprising a polynucleotide encoding said polypeptide with lactose binding α-1,2-fucosyltransferase activity wherein said sequence is foreign to the host cell and is integrated in the genome of the host cell, or
   ii) containing a vector comprising a polynucleotide encoding said polypeptide, wherein the polynucleotide being operably linked to control sequences recognized by a host cell transformed with the vector.
14. [CS13][NL-I14][CS15]Cell[NL-I16] according to any one of embodiment 12 or 13, wherein said cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal; preferably the cell is an Escherichia coli cell.
15. Host cell according to any one of embodiment 12 to 14, characterized in that the host cell is a cell of a bacterium, preferably of an Escherichia coli strain, more preferably of an Escherichia coli strain which is a K12 strain, even more preferably the Escherichia coli K12 strain is Escherichia coli MG1655.
16. Host cell according to any one of embodiment 12 to 14, characterized in that the host cell is a yeast cell.
17. Host cell according to any one of embodiment 12 to 16, characterized in that the polynucleotide encoding the polypeptide with lactose binding alpha-1,2-fucosyltransferase activity is adapted to the codon usage of the respective host cell.
18. Method for the production of α-1,2-fucosyllactose, comprising the steps of:
   a) providing a cell according to any one of embodiments 12 to 17,
   b) cultivating the cell in a medium under conditions permissive for the production of α-1,2-fucosyllactose,
   c) optionally, separating said α-1,2-fucosyllactose from the cultivation.
19. Use of a host cell according to any one of embodiment 12 to 17 for the production of α-1,2-fucosyllactose.
20. A microorganism heterologously expressing a lactose binding alpha-1,2-fucosyltransferase polypeptide wherein said polypeptide
   i) comprises an amino acid sequence encoding a conserved domain G-Y-[F/Y]-Q-[N/S] (SEQ ID NO: 72) and an amino acid sequence encoding a conserved domain (X, no K/V)XXXX[I/L]H[I/L]R[R/L]GD[F/Y](X, no C/M) (SEQ ID NO: 74) wherein X can be any distinct amino acid excluding a lysine and a valine residue from the first position of said domain and excluding a cysteine and a methionine residue from the last position of said domain, and/or
   ii) is selected from the group consisting of:
      i) any one of SEQ ID NO 01, 02, 03, 04, 05, 06, 07, 09, 10, 11, 12, 13, 14, 15, 16, 17, 19, 21, 22, 25, 26, 29, 33, 34, 35, 36, 37, 38, 39, 40, 41, 43, 44, 45, 46, 47, 48, 50, 51, 52, 53, 54, 55, 56, 57, 58, 61, 62, 63, 66, 67 or 76 of the attached sequence listing, or
      ii) an amino acid sequence having 80% or more sequence identity to the full-length amino acid sequence of any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79 of the attached sequence listing, or
      iii) an amino acid sequence of an allelic variant of an amino acid sequence shown in any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, or
      iv) an amino acid sequence of an ortholog of an amino acid sequence shown in any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79, and
      v) a functional fragment of an amino acid sequence shown in any one of SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 75, 76, 77, 78 or 79,
   preferably said polypeptide belongs to the glycosyl transferase 11 (GT11) family. [CS17]
21. Use of a microorganism according to embodiment 20 for the production of alpha-1,2-fucosyllactose.
22. The method of any one of embodiments 1 to 11, 13, 19 or 21 further comprising a step of separating said alpha-1,2-fucosyllactose from the host cell or the medium of its growth.
23, The method of any one of embodiments 1 to 11, 18, 19, 21 or 22, wherein said separation comprises at least one of the following steps: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography and/or gel filtration, ligand exchange chromatography.
24. The method of any one of embodiments 1 to 11, 18, 19, 21 to 23, further comprising purification of alpha-1,2-fucosyllactose.
25. The method of embodiment 24, wherein said purification of said alpha-1,2-fucosyllactose comprises at least one of the following steps: use of activated charcoal or carbon, use of charcoal, nanofiltration, ultrafiltration or ion exchange, use of alcohols, use of aqueous alcohol mixtures, crystallization, evaporation, precipitation, drying, spray drying or lyophilization.
26. The method of any one of embodiment 1 to 11, 21 to 25, wherein the polypeptide is produced in a fungal, yeast, bacterial, insect, animal or plant expression system.
27. The method of embodiment 26, wherein the host cell is a cell of a bacterium, preferably of an Escherichia coli strain, more preferably of an Escherichia coli strain which is a K12 strain, even more preferably the Escherichia coli K12 strain is Escherichia coli MG1655.
28. The method of embodiment 26, wherein the host cell is a yeast cell.
29. The method of any one of embodiments 1 to 11, 18, 19, 21 to 28, wherein the lactose concentration in the culture medium ranges from 50 to 150 g/L.
30. The method of any one of embodiments 1 to 11, 18, 19, 21 to 29 wherein the final concentration of 2′-fucosyllactose ranges between 70 g/L to 200 g/L.
31. Use of a polypeptide as described in the method of any one of embodiment 1 or 10 for the production of α-1,2-fucosyllactose.
32, A method for the production of α-1,2-fucosyllactose, the method comprising the steps of:
   a) providing a polypeptide with α-1,2-fucosyltransferase activity and with the ability to use lactose as acceptor substrate
   b) contacting the polypeptide with α-1,2-fucosyltransferase activity of step a) with a mixture comprising GDP-fucose as donor substrate, and lactose as acceptor substrate, under conditions where the polypeptide catalyses the transfer of a fucose residue from the donor substrate to the acceptor substrate,
   thereby producing α-1,2-fucosyllactose,
   c) wherein said catalysis results in a diFL concentration to 2′fucosyllactose concentration ratio of less than 1:5
   d) optionally separating said α-1,2-fucosyllactose.
33. A method for the production of α-1,2-fucosyllactose, the method comprising the steps of:
   a) providing a polypeptide with α-1,2-fucosyltransferase activity and with the ability to use lactose as acceptor substrate
   b) contacting the polypeptide with α-1,2-fucosyltransferase activity of step a) with a mixture comprising GDP-fucose as donor substrate, and lactose as acceptor substrate, under conditions where the polypeptide catalyses the transfer of a fucose residue from the donor substrate to the acceptor substrate, thereby producing α-1,2-fucosyllactose,
   c) wherein said catalysis results in a 2′fucosyllactose purity of 80% or more.
   d) optionally separating said α-1,2-fucosyllactose.
34. Method for the production of 2′-fucosyllactose[CS18] comprising at least one of the following steps:
   i) adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter)[NL19], preferably in a continuous manner, and preferably so that the final volume of the culture medium is not more than three-fold, preferably not more than two-fold, more preferably less than 2-fold of the volume of the culture medium before the addition of said lactose feed;
   ii) adding to the culture medium a GDP-fucose feed at a concentration enabling the host cell to synthesize 2′-fucosyllactose with a diFL concentration to 2′fucosyllactose concentration ratio of less than 1:5, preferably in a continuous manner and preferably so that the final volume of the culture medium is not more than three-fold, preferably not more than two-fold, more preferably less than 2-fold of the volume of the culture medium before the addition of said GDP-fucose feed;
   iii) adding to the culture medium a carbon-based substrate feed at a concentration enabling the host cell to synthesize GDP-fucose at an optimal concentration for 2′fucosyllactose synthesis with a diFL concentration to 2′fucosyllactose concentration ratio of less than 1:5, preferably in a continuous manner;
   iv) adding to the culture medium a carbon-based substrate feed at a concentration enabling the host cell to internally synthesize lactose at an optimal concentration for 2′fucosyllactose synthesis;
   v) adding a lactose feed, a GDP-fucose feed and/or a carbon-based substrate feed in a continuous manner to the culture medium over the course of 1 day, 2 days, 3 days, 4 days, 5 days by means of at least one feeding solution;
   vi) adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
   vii) said method resulting in a 2′-fucosyllactose concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium and preferably a diFL concentration to 2′FL concentration ratio lower than 1:5, more preferably 1:20, even more preferably 1:40.
35. Method for the production of 2′-fucosyllactose comprising at least one of the following steps:
   i) adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter)[NL20], preferably in a continuous manner, and preferably so that the final volume of the culture medium is not more than three-fold, preferably not more than two-fold, more preferably less than 2-fold of the volume of the culture medium before the addition of said lactose feed;
   ii) adding to the culture medium a GDP-fucose feed at a concentration enabling the host cell to synthesize 2′-fucosyllactose with a purity of at least 80%, preferably in a continuous manner and preferably so that the final volume of the culture medium is not more than three-fold, preferably not more than two-fold, more preferably less than 2-fold of the volume of the culture medium before the addition of said GDP-fucose feed;
   iii) adding to the culture medium a carbon-based substrate feed at a concentration enabling the host cell to synthesize GDP-fucose at an optimal concentration for 2′fucosyllactose synthesis with a purity of at least 80%, preferably in a continuous manner;
   iv) adding to the culture medium a carbon-based substrate feed at a concentration enabling the host cell to internally synthesize lactose at an optimal concentration for 2′fucosyllactose synthesis;
   v) adding a lactose feed, a GDP-fucose feed and/or a carbon-based substrate feed in a continuous manner to the culture medium over the course of 1 day, 2 days, 3 days, 4 days, 5 days by means of at least one feeding solution;
   vi) adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
   vii) said method resulting in a 2′-fucosyllactose concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium and preferably a 2′FL purity of 80% or more.

The following drawings and examples will serve as further illustration and clarification of the present invention and are not intended to be limiting.

### BRIEF DESCRIPTION OF THE DRAWINGS

FIG. 1 shows the normalized production of 2′FL (upperpanel) and diFL (lower panel) in g product/g biomass in relative percentages of mutant strains expressing a different alpha-1,2-fucosyltransferase (with either SEQ ID NO 01, 02, 03, 04 or 05) from a transcriptional unit built with promoter-UTR combination P12U2 (De Mey et al., BMC Biotechnology, 2007) after 72 hours of cultivation in minimal medium supplemented with 20 g/L lactose as described in Example 2. The 2′FL and diFL production in each strain is normalized to the 2′FL and diFL production levels, respectively, obtained in a reference strain expressing the prior art HpFutC gene with SEQ ID NO 71 (indicated by the dashed horizontal line).

FIG. 2 shows the normalized 2′FL production (2FL) and growth speed (mu) in relative percentages (%) of mutant strains expressing a different alpha-1,2-fucosyltransferase (with either SEQ ID NO 01 or 04) from a transcriptional unit built with promoter-UTR combination P12U2 (De Mey et al., BMC Biotechnology, 2007) after 72 hours of cultivation in minimal medium supplemented with 20 g/L lactose as described in Example 2. The 2′FL production levels and the growth speed of each strain are normalized to the 2′FL production levels and the growth speed obtained in a reference strain expressing the prior art HpFutC gene with SEQ ID NO 71 (indicated by the dashed horizontal line).

FIG. 3 shows the 2′FL titers (g/L) obtained in a mutant strain expressing the alpha-1,2-fucosyltransferase with SEQ ID NO 03 from a transcriptional unit built with promoter-UTR combination P12U2 (De Mey et al., BMC Biotechnology, 2007) after 72 hours of cultivation in minimal medium supplemented with different concentrations of lactose (2.5, 5, 10, 20, 45 and 70 g/L) as described in Example 2.

FIG. 4 shows the normalized 2′FL production (2FL) and growth speed (mu) in relative percentages (%) of mutant strains expressing the alpha-1,2-fucosyltransferase with SEQ ID NO 03 from four different transcriptional units (TU) after 72 hours of cultivation in minimal medium supplemented with 20 g/L lactose as described in Example 2. The transcriptional units consist of promoter-UTR combinations P05U14 (TU 01), P05U38 (TU 02), P10U13 (TU 03) and P31U17 (TU 04) as described by De Mey et al. (BMC Biotechnology, 2007). For each TU tested, the 2′FL production levels and the growth speed of each strain are normalized to the 2′FL production levels and the growth speed respectively obtained in a reference strain expressing the prior art HpFutC gene with SEQ ID NO 71 from the same transcriptional unit (indicated by the dashed horizontal line).

FIG. 5 shows the diFL concentration to 2′FL concentration ratio obtained in whole broth samples at the end of a fed-batch fermentation process with strains expressing the alpha-1,2-fucosyltransferase with SEQ ID NO 01, 02 or 71 as described in Example 2.

FIG. 6 shows the relative 2′FL, diFL and lactose concentration (in %) at various timepoints during fermentation processes with strains expressing the alpha-1,2-fucosyltransferase with SEQ ID NO 01, 03, 04 or 71 as described in Example 2.

### EXAMPLES

### Example 1: Identification of Glycosyl Transferase 11 Proteins

An HMM is a probabilistic model called profile hidden Markov models. It characterizes a set of aligned proteins into a position-specific scoring system. Amino acids are given a score at each position in the sequence alignment according to the frequency by which they occur (Eddy, S. R. 1998. Profile hidden Markov models. Bioinformatics. 14: 755-63). HMMs have wide utility, as is clear from the numerous databases that use this method for protein classification, including Pfam, InterPro, SMART, TIGRFAM, PIRSF, PANTHER, SFLD, Superfamily and Gene3D. HMMsearch from the HMMER package (http://hmmer.org/) can use this HMM to search sequence databases for sequence homologs. Sequence databases that can be used are for example, but not limited to: the NCBI nr Protein Database (NR; https://www.ncbi.nlm.nih.gov/protein), UniProt Knowledgebase (UniProtKB, https://www.uniprot.org/help/uniprotkb) and the SWISS-PROT database (https://web.expasy.org/docs/swiss-prot_guideline.html). The Glycosyl transferase 11 PF01531 domain was obtained from the Pfam v 32.0 database as released in September 2018 via https://pfam.xfam.org/search#tabview=tab1 in ‘Curation & model’. HMMsearch with this model to the protein databases identified new family members (see Table 1). EMBOSS Backtranseq (https://www.ebi.ac.uk/Tools/st/emboss_backtranseq/) can be used to translate the obtained amino acid sequence into a nucleotide sequence.

TABLE 1
List of alpha 1,2-fucosyltransferases together with the prior
art alpha 1,2-fucosyltransferase HpFutC from H. pylori (SEQ
ID NO 71), wherein SEQ ID NOs 1, 2, 3, 4, 5, 6, 7, 9, 10, 11,
12, 13, 14, 15, 16, 17, 19, 21, 22, 25, 26, 29, 33, 34, 35, 36,
37, 38, 39, 40, 41, 43, 44, 45, 46, 47, 48, 50, 51, 52, 53, 54,
55, 56, 57, 58, 61, 62, 63, 66, 67 and 76 have both the conserved
domain G-Y-[F/Y]-Q-[N/S] (SEQ ID NO: 72) and the conserved
domain (X, no K/V)XXXX[I/L]H[I/L]R[R/L]GD[F/Y](X,
no C/M) (SEQ ID NO: 74), wherein X can be any distinct amino
acid excluding a lysine and a valine residue from the first position
of said domain and excluding a cysteine and a methionine residue
from the last position of said domain.

| SEQ | | |
|---|---|---|
| ID NO | Organism | Country origin |
| 1 | Akkermansia muciniphila | Human |
| 2 | Porphyromonas catoniae | Unknown |
| 3 | Helicobacter sp. MIT 01-6242 | USA |
| 4 | Capnocytophaga canis | Unknown |
| 5 | Capnocytophaga leadbetteri | Finland |
| 6 | Helicobacter sp. MIT 01-6242 | USA |
| 7 | Kingella denitrificans | Human |
| 8 | Pseudoalteromonas distincta | Unknown |
| 9 | Porphyromonas catoniae F0037 | Unknown |
| 10 | Campylobacter mucosalis | UK |
| 11 | Pedobacter kyungheensis | South Korea |
| 12 | Noviherbaspirillum autotrophicum | Japan |
| 13 | Microbacterium trichothecenolyticum | Unknown |
| 14 | Candidatus Symbiothrix dinenymphae | Japan |
| 15 | Pedobacter sp. | Switzerland |
| 16 | Chryseobacterium sp. | Switzerland |
| 17 | Microbacterium sp. | Germany |
| 18 | Ramazzottius varieornatus | Japan |
| 19 | Candidatus Wolfebacteria bacterium | USA |
| 20 | Candidatus Yanofskybacteria bacterium | USA |
| 21 | Spirochaetes bacterium | USA |
| 22 | Pedobacter soli | South Korea |
| 23 | Bacteroidales bacterium KHT7 | Unknown |
| 24 | Butyrivibrio sp. TB | Unknown |
| 25 | Butyrivibrio fibrisolvens | Unknown |
| 26 | Candidatus Magasanikbacteria bacterium | USA |
| 27 | Butyrivibrio fibrisolvens | Unknown |
| 28 | Chryseobacterium scophthalmum | UK |
| 29 | Akkermansia sp. 54_46 | Unknown |
| 30 | Lingula unguis | Japan |
| 31 | Lingula unguis | Japan |
| 32 | [Flexibacter] sp. ATCC 35103 | USA |
| 33 | Bacteroidetes bacterium ADurb. Bin174 | USA |
| 34 | Pedobacter sp. AJM | USA |
| 35 | Candidatus Planktophila lacus | Switzerland |
| 36 | Rhizobiales bacterium PAR1 | UK |
| 37 | Helicobacter sp. 11S02629-2 | Netherlands |
| 38 | Candidatus Magasanikbacteria bacterium | USA |
| 39 | Candidatus Magasanikbacteria bacterium | USA |
| 40 | Diaminobutyricimonas aerilata | South Korea |
| 41 | Flavobacterium magnum | South Korea |
| 42 | Litoreibacter ponti | South Korea |
| 43 | Dysgonomonas alginatilytica | Japan |
| 44 | Campylobacter hyointestinalis subsp. lawsonii | USA |
| 45 | Phycisphaerales bacterium | Unknown |
| 46 | Akkermansia sp. | Unknown |
| 47 | Helicobacter sp. MIT 17-337 | USA |
| 48 | Pedobacter sp. G11 | USA |
| 49 | Empedobacter brevis | Unknown |
| 50 | Akkermansia muciniphila | South Korea |
| 51 | Akkermansia muciniphila | Human |
| 52 | Akkermansia muciniphila | USA |
| 53 | Chitinophagaceae bacterium | UK |
| 54 | Chitinophagaceae bacterium | UK |
| 55 | Proteobacteria bacterium | UK |
| 56 | Flaviaesturariibacter sp. 17J68-12 | South Korea |
| 57 | Pedobacter sp. AR-2-6 | Norway |
| 58 | Helicobacter jaachi | USA |
| 59 | Helicobacter japonicus | USA |
| 60 | Klebsiella pneumoniae | Unknown |
| 61 | Dysgonomonas capnocytophagoides | USA |
| 62 | Flavobacterium sp. | UK |
| 63 | Pedobacter sp. | UK |
| 64 | Hoeflea phototrophica | Germany |
| 65 | Subdoligranulum variabile | Denmark |
| 66 | Crassostrea gigas | Unknown |
| 67 | Crassostrea gigas | Unknown |
| 68 | uncultured bacterium | USA |
| 69 | Clostridium sp. CAG:510 | Unknown |
| 70 | Helobdella robusta | USA |
| 71 | Helicobacter pylori | Australia |
| 75 | Akkermansia sp. KLEI798 | Unknown |
| 76 | Akkermansia muciniphila | Netherlands |
| 77 | Capnocytophaga gingivalis ATCC 33624 | Unknown |
| 78 | Helicobacter sp. CLO-3 | USA |
| 79 | Helicobacter sp. MIT 17-337 | USA |

### Example 2: Materials & Methods Escherichia coli

Media

The Luria Broth (LB) medium consisted of 1% tryptone peptone (Difco, Erembodegem, Belgium), 0.5% yeast extract (Difco) and 0.5% sodium chloride (VWR, Leuven, Belgium). The minimal medium for the growth experiments contained 2.00 g/L NH4Cl, 5.00 g/L (NH4)2SO4, 2.993 g/L KH2PO4, 7.315 g/L K2HPO4, 8.372 g/L MOPS, 0.5 g/L NaCl, 0.5 g/L MgSO4.7H2O, 14.26 g/L sucrose or another carbon source when specified in the examples, 1 ml/L vitamin solution, 100 μl/L molybdate solution, and 1 mL/L selenium solution. The medium was set to a pH of 7 with 1M KOH. Vitamin solution consisted of 3.6 g/L FeCl2·4H2O, 5 g/L CaCl2·2H2O, 1.3 g/L MnCl2·2H2O, 0.38 g/L CuCl2·2H2O, 0.5 g/L CoCl2·6H2O, 0.94 g/L ZnCl2, 0.0311 g/L H3BO4, 0.4 g/L Na2EDTA·2H2O and 1.01 g/L thiamine·HCl. The molybdate solution contained 0.967 g/L NaMoO4·2H2O. The selenium solution contained 42 g/L Seo2.

The minimal medium for fermentations contained 6.75 g/L NH4Cl, 1.25 g/L (NH4)2SO4, 2.93 g/L KH2PO4 and 7.31 g/L KH2PO4, 0.5 g/L NaCl, 0.5 g/L MgSO4.7H2O, 14.26 g/L sucrose, 1 mL/L vitamin solution, 100 μL/L molybdate solution, and 1 mL/L selenium solution with the same composition as described above. Complex medium was sterilized by autoclaving (121° C., 21′) and minimal medium by filtration (0.22 μm Sartorius). When necessary, the medium was made selective by adding an antibiotic (e.g. chloramphenicol (20 mg/L), carbenicillin (100 mg/L), spectinomycin (40 mg/L) and/or kanamycin (50 mg/L)).

Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007).

Plasmids were maintained in the host E. coli DH5alpha (F⁻, phi80dlacZΔM15, Δ(lacZYA-argF) U169, deoR, recA1, endA1, hsdR17(rk⁻, mk⁺), phoA, supE44, lambda⁻, thi-1, gyrA96, relA1) bought from Invitrogen.

Strains and Mutations

Escherichia coli K12 MG1655 [lambda⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

Transformants carrying a Red helper plasmid pKD46 were grown in 10 mL LB media with ampicillin, (100 mg/L) and L-arabinose (10 mM) at 30° C. to an OD_{600 nm} of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600 Ω, 25 μFD, and 250 volts).

After electroporation, cells were added to 1 ml LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity.

The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with DpnI, repurified from an agarose gel, and suspended in elution buffer (5 mM Tris, pH 8.0).

The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers (Fw/Rv-gene-out).

For 2′FL production, the mutant strains derived from E. coli K12 MG1655 have knock-outs of the genes lacZ, lacY, lacA, glgC, agp, pfkA, pfkB, pgi, arcA, icIR, wcaJ, pgi, Ion and thyA and additionally genomic knock-ins of constitutive expression constructs containing the E. coli lacY gene, a fructose kinase gene (frk) originating from Zymomonas mobilis and a sucrose phosphorylase (SP) originating from Bifidobacterium adolescentis. These genetic modifications are also described in WO2016075243 and WO2012007481. GDP-fucose production can additionally be optimized comprising genomic knock-ins of constitutive transcriptional units for the E. coli genes manA, manB, manC, gmd and fcl. GDP-fucose production can also be obtained by genomic knock-outs of the E. coli fucK and fucI genes and genomic knock-ins of constitutive transcriptional units containing the fucose permease (fucP) from E. coli and the bifunctional fucose kinase/fucose-1-phosphate guanylyltransferase (fkp) from Bacteroides fragilis. In addition, an alpha-1,2-fucosyltransferase expression plasmid having a constitutive transcriptional unit for SEQ ID NO 01, 02, 03, 04, 05 or 71 is added to the strains.

All constitutive promoters and UTRs originate from the libraries described by De Mey et al. (BMC Biotechnology, 2007) and Mutalik et al. (Nat. Methods 2013, No. 10, 354-360). All genes were ordered synthetically at Twist Bioscience (twistbioscience.com) or IDT (eu.idtdna.com) and the codon usage was adapted using the tools of the supplier.

All strains are stored in cryovials at −80° C. (overnight LB culture mixed in a 1:1 ratio with 70% glycerol).

Cultivation Conditions

A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL minimal medium by diluting 400×. These final 96-well culture plates were then incubated at 37° C. on an orbital shaker at 800 rpm for 72 h, or shorter, or longer. At the end of the cultivation experiment samples were taken from each well to measure sugar concentrations in the broth supernatant (extracellular sugar concentrations, after spinning down the cells), or by boiling the culture broth for 15 min at 90° C. before spinning down the cells (=whole broth measurements, average of intra- and extracellular sugar concentrations).

A preculture for the bioreactor was started from an entire 1 mL cryovial of a certain strain, inoculated in 250 mL or 500 mL of minimal medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2SO4 and 20% NH40H. The exhaust gas was cooled. 10% solution of silicone antifoaming agent was added when foaming raised during the fermentation.

Optical Density

Cell density of the cultures was frequently monitored by measuring optical density at 600 nm (Implen Nanophotometer NP80, Westburg, Belgium or with a Spark 10M microplate reader, Tecan, Switzerland).

Growth Rate/Speed Measurement

The maximal growth rate (μMax) was calculated based on the observed optical densities at 600 nm using the R package grofit.

Liquid Chromatography

Standards for 2′fucosyllactose and difucosyllactose were synthetized in house. Other standards such as but not limited to lactose, sucrose, glucose, fructose, fucose were purchased from Sigma.

Carbohydrates were analyzed via a HPLC-RI (Waters, USA) method, whereby RI (Refractive Index) detects the change in the refraction index of a mobile phase when containing a sample. The sugars were separated in an isocratic flow using an X-Bridge column (Waters X-bridge HPLC column, USA) and a mobile phase containing 75 ml acetonitrile and 25 ml Ultrapure water and 0.15 ml triethylamine. The column size was 4.6×150 mm with 3.5 μm particle size. The temperature of the column was set at 35° C. and the pump flow rate was 1 mL/min.

### Example 3: Materials & Methods Saccharomyces cerevisiae

Media

Strains are grown on Synthetic Defined yeast medium with Complete Supplement Mixture (SD CSM) or CSM drop-out (SD CSM-Ura) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura (MP Biomedicals).

Strains

Saccharomyces cerevisiae BY4742 created by Brachmann et al. (Yeast (1998) 14:115-32) was used available in the Euroscarf culture collection. All mutant strains were created by homologous recombination or plasmid transformation using the method of Gietz (Yeast 11:355-360, 1995). Kluyveromyces marxianus lactis is available at the LMG culture collection (Ghent, Belgium).

Plasmids

Yeast expression plasmid p2a_2μ (Chan 2013 (Plasmid 70 (2013) 2-17)) was used for expression of foreign genes in Saccharomyces cerevisiae. This plasmid contains an ampicillin resistance gene and a bacterial origin of replication to allow for selection and maintenance in E. coli. The plasmid further contains the 2μ yeast on and the Ura3 selection marker for selection and maintenance in yeast. Next, this plasmid can be modified to p2a_2μ_fl to contain a lactose permease (for example LAC12 from Kluyveromyces lactis), a GDP-mannose 4,6-dehydratase (such as Gmd from E. coli) and a GDP-L-fucose synthase (such as fcl from E. coli).

Yeast expression plasmids p2a_2μ_fl_2ft are based on p2a_2μ_fl but modified in a way that also SEQ ID NO 01, 02, 03, 04, 05, 06, 07, 09 or 71 are expressed. Preferably but not necessarily, the fucosyltransferase proteins are N-terminally fused to a SUMOstar tag (e.g. obtained from pYSUMOstar, Life Sensors, Malvern, PA) to enhance the solubility of the fucosyltransferase enzymes.

Plasmids were maintained in the host E. coli DH5alpha (F⁻, phi80dlacZdeltaM15, delta(lacZYA-argF)U169, deoR, recA1, endA1, hsdR17(rk⁻, mk⁺), phoA, supE44, lambda⁻, thi-1, gyrA96, relA1) bought from Invitrogen.

Gene Expression Promoters

Genes are expressed using synthetic constitutive promoters, as described by Blazeck (Biotechnology and Bioengineering, Vol. 109, No. 11, 2012).

Heterologous and Homologous Expression

Genes that needed to be expressed, be it from a plasmid or from the genome, were synthetically synthetized by Twist Biosciences (San Francisco, USA). Expression could be further facilitated by optimizing the codon usage to the codon usage of the expression host. Genes were optimized using the tools of the supplier.

Cultivations Conditions

In general, yeast strains were initially grown on SD CSM plates to obtain single colonies. These plates were grown for 2-3 days at 30° C.

Starting from a single colony, a preculture was grown overnight in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm. The use of an inducer is not required as all genes are constitutively expressed.

### Example 4: Evaluation of Potential Lactose-Binding Alpha-1,2-Fucosyltransferase Enzymes Incorporated in Escherichia coli

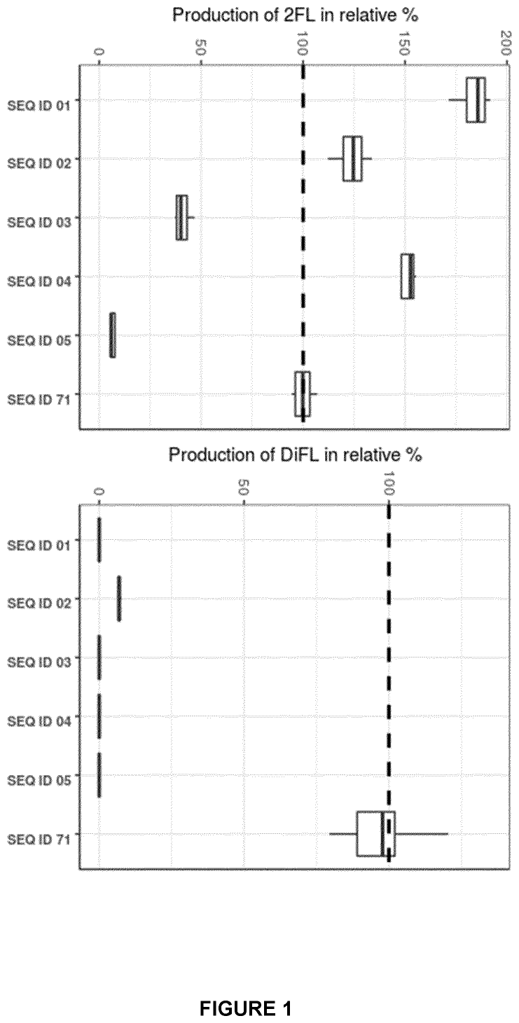

The alpha-1,2-fucosyltransferase enzymes with SEQ ID NO 01, 02, 03, 04 and 05 identified in Example 1 were evaluated to produce 2-fucosyllactose (2′FL) from GDP-fucose and lactose in mutant E. coli strains. Mutant strains expressing said alpha-1,2-fucosyltransferases from a transcriptional unit built with promoter-UTR combination P12U2 (De Mey et al., BMC Biotechnology, 2007) were analysed in a growth experiment according to the cultivation conditions provided in Example 2. Each mutant E. coli strain was grown in multiple wells of a 96-well plate. FIG. 1 (upper panel) shows the normalized production of 2′FL (g product/g biomass in relative percentages) obtained in a growth experiment of the strains successfully expressing various alpha-1,2-fucosyltransferases from the same transcriptional unit with 20 g/L lactose in the minimal medium. All datapoints were normalized to the 2′FL production obtained with a reference strain expressing the prior art alpha-1,2-fucosyltransferase HpFutC with SEQ ID NO 71 from a transcriptional unit with the same P12U2 promoter-UTR combination, indicated by the dashed horizontal line. The experiment identified all newly identified polypeptides to have lactose binding 2-fucosyltransferase activity. Surprisingly, the polypeptides with SEQ ID NO 01, 02 and 04 were identified to have better lactose binding 2-fucosyltransferase activity than the prior art alpha-1,2-fucosyltransferase HpFutC with SEQ ID NO 71 in these conditions tested. The experiment also demonstrated that the polypeptides with SEQ ID NOs 01, 03, 04 and 05 did not produce diFL unlike the prior art enzyme with SEQ ID NO 71. The polypeptide with SEQ ID NO 02 produced diFL but this diFL production was very minimal compared to the diFL production measured with the prior art HpFutC enzyme with SEQ ID NO 71 (FIG. 1, lower panel). The diFL concentration to 2′FL concentration ratio measured in the strain expressing the polypeptide with SEQ ID NO 02 was 1:40.

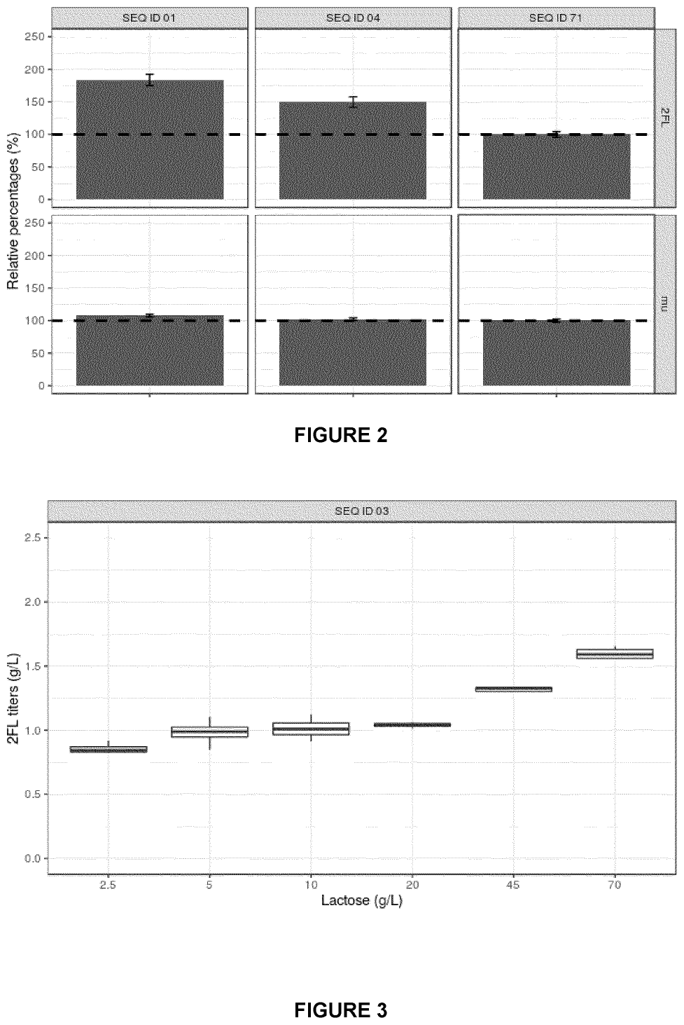

The experiment also showed that the higher production of 2′FL in the mutant strains expressing the polypeptides with SEQ ID NO 01 or 04 did not affect the growth speed of these strains when compared to the reference strain expressing the prior art alpha-1,2-fucosyltransferase HpFutC with SEQ ID NO 71 (FIG. 2).

### Example 5: Evaluation of an E. coli Strain Expressing a Lactose-Binding Alpha-1,2-Fucosyltransferase Enzyme when Tested on Different Lactose Concentrations

In a next experiment, the mutant E. coli strain expressing the alpha-1,2-fucosyltransferase enzyme with SEQ ID NO 03 from a transcriptional unit built with the P12U2 promoter-UTR combination (De Mey et al., BMC Biotechnology, 2007) as described in Example 4 was evaluated for 2′FL and diFL production on minimal medium containing low to high lactose concentrations. A growth experiment was performed according to the cultivation conditions provided in Example 2. For each condition, the mutant E. coli strain was grown in multiple wells of a 96-well plate.

FIG. 3 shows the 2′FL titers (g/L) obtained in this experiment. [CS21][NL-I22][23][24] This figure demonstrates that the polypeptide with SEQ ID NO 03 has lactose binding 2-fucosyltransferase activity in all conditions tested and is able to produce 2′FL from various lactose concentrations. In all tested conditions the strain did not produce diFL (Results not shown).

### Example 6: Evaluation of an E. coli Strain Expressing a Lactose-Binding Alpha-1,2-Fucosyltransferase Enzyme from Different Transcriptional Units

In a next experiment, several mutant E. coli strains expressing the alpha-1,2-fucosyltransferase polypeptide with SEQ ID NO 03 from transcriptional units (TU) that were different compared to those used in previous examples were evaluated for 2′FL and diFL production. The transcriptional units used consisted of promoter-UTR combinations P05U14 (TU 01), P05U38 (TU 02), P10U13 (TU 03) and P31U17 (TU 04) respectively as described by De Mey et al. (BMC Biotechnology, 2007). A growth experiment was performed according to the cultivation conditions provided in Example 2. Each mutant E. coli strain was grown in multiple wells of a 96-well plate.

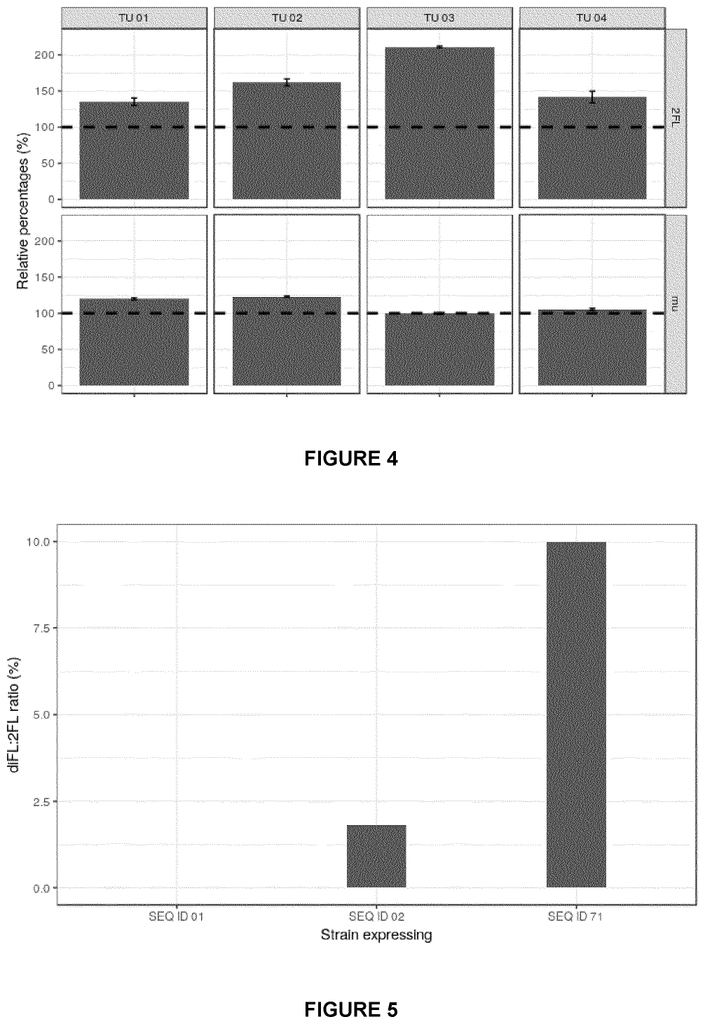

FIG. 4 shows the normalized 2′FL production (g product/g biomass) and growth speed in relative percentages obtained in this experiment. For each strain and thus for each transcriptional unit tested, the datapoints for 2′FL production or growth speed were normalized to the 2′FL production or growth speed respectively obtained for a reference strain expressing the prior art alpha 1,2-fucosyltransferase HpFutC with SEQ ID NO 71 from the identical transcriptional unit (indicated by the dashed horizontal line). This experiment demonstrated the strains all produced 2′FL without diFL production in all tested conditions. In addition, one can increase the 2′FL production in a mutant strain depending on the transcriptional unit used, with limited effect on the strain's growth speed. The four transcriptional units used here in the mutant strains to express the polypeptide with SEQ ID NO 03 also resulted in a higher expression of said polypeptide compared to when using a transcriptional unit built of P12U2 as used in Example 4, leading to more 2′FL production.

### Example 7: Production of 2′Fucosyllactose with an E. coli Strain Expressing a Combination of Lactose-Binding Alpha-1,2-Fucosyltransferase Enzymes

In another experiment, several mutant E. coli strains expressing a combination of two or more of the alpha-1,2-fucosyltransferase polypeptides with SEQ ID NO 01, 02, 03, 04, 05, 06, 07 or 09 were evaluated for 2′FL and diFL production. A growth experiment was performed according to the cultivation conditions provided in Example 2. Each mutant E. coli strain was grown in multiple wells of a 96-well plate. The experiment showed that a single strain expressing combinations of at least two alpha-1,2-fucosyltransferase polypeptides is able to produce 2′FL and in higher concentrations than when only a single polypeptide is expressed per strain.

### Example 8: Production of 2′Fucosyllactose in Saccharomyces cerevisiae Using Various Lactose Binding Alpha-1,2-Fucosyltransferase Enzymes

Another example provides use of a eukaryotic organism, in the form of Saccharomyces cerevisiae, for the invention. Using the strains, plasmids and methods as described in Example 3, strains are created that express SEQ ID NO 01, 02, 03, 04, 05, 06, 07, 09 or 71. On top of that, further modifications are made in order to produce 2′fucosyllactose. These modifications comprise the addition of a lactose permease, a GDP-mannose 4,6-dehydratase and a GDP-L-fucose synthase. The preferred lactose permease is the KILAC12 gene from Kluyveromyces lactis (WO 2016/075243). The preferred GDP-mannose 4,6-dehydratase and the GDP-L-fucose synthase are respectively gmd and fcl from Escherichia coli.

These strains are capable of growing on glucose or glycerol as carbon source, converting the carbon source into GDP-L-fucose, taking up lactose, and producing 2′fucosyllactose using GDP-L-fucose and lactose as substrates for the enzymes represented by SEQ ID NOs 01, 02, 03, 04, 05, 06, 07, 09 and 71 with SEQ ID NO 71 as reference. Preculture of said strains are made in 5 mL of the synthetic defined medium SD-CSM containing 22 g/L glucose and grown at 30° C. as described in Example 3. These precultures are inoculated in 25 mL medium in a shake flask with 10 g/L sucrose as sole carbon source and grown at 30° C. Regular samples are taken and the production of 2′fucosyllactose and di-fucosyllactose is measured as described in Example 2.

### Example 9: Evaluation of Escherichia coli Strains Expressing Various Lactose Binding Alpha-1,2-Fucosyltransferase Enzymes in a Batch Fermentation

Batch fermentations at bioreactor scale were performed to evaluate strains, derived from the mutant E. coli K12 MG1655 strain background as described in Example 2, expressing the alpha-1,2-fucosyltransferase enzymes with SEQ ID NO NOs 01, 02, 03, 04, 05, 06, 07, 09 or 71 with SEQ ID NO 71 as reference. The bioreactor runs were performed as described in Example 2. In these examples, sucrose was used as a carbon source. Lactose was added in the batch medium at 90 g/L as a precursor for 2′FL formation. Regular samples are taken and the production of 2′fucosyllactose and di-fucosyllactose is measured as described in Example 2.

### Example 10: 2′Fucosyllactose Production with Different Lactose Concentrations

A fermentation process as described in Example 2 was performed wherein the lactose concentration in the culture medium ranges from 50 to 150 g/L. Said lactose is converted during the process into 2′fucosyllactose until minor amounts of lactose is left. The final ratio lactose to 2′fucosyllactose may be manipulated during this process by stopping the process earlier (higher lactose to 2′fucosyllactose ratio) or later (lower lactose to 2′fucosyllactose ratio). The lactose concentration may be increased in the vessel by feeding high concentrations of lactose solution with or without another carbon source to the bioreactor. Said lactose feed contains lactose concentrations between 100 and 700 g/L and is kept at a temperature so that the lactose is kept soluble at a pH below or equal to 6 to avoid lactulose formation during the process, a standard method used in the dairy industry. The final concentrations of 2′fucosyllactose reached in such a production process ranges between 70 g/L when lower lactose concentrations are used and 200 g/L or higher when high lactose concentrations are used in the process as described above. The 2′FL titers that can be obtained in such a fed-batch fermentation process are much higher compared to batch growth experiments in shake flasks or multiwell plates. In a well-controlled bioreactor, additional carbon source is constantly added during the fed-batch and much higher biomass concentrations can be obtained (better aeration and less acidification compared to multiwell plate batch experiments). The final difucosyllactose concentration to 2′FL concentration ratio obtained in such a process with strains producing 2′FL from SEQ ID NO 01 or 02 e.g. is 0 or 1:55, respectively, which is much smaller than the diFL to 2′FL ratio of 1:6 (a ratio of 0,164) at a lactose concentration near 0 g/L obtained in a similar process with a reference strain producing 2′FL from SEQ ID NO 71 (FIG. 5). For this reference strain the ratios were measured at different residual lactose concentrations and this strain reaches diFL to 2′FL ratios of 1:8 at 3 g/L residual lactose, 1:9 at 5 g/L residual lactose, 1:11 at 8 g/L residual lactose, 1:12 at 14 g/L residual lactose and 1:22 at 21 g/L residual lactose. The resulting 2′FL purity ranges between 76% and 86% wherein diFL forms a substantial fraction of the remaining 24 to 14%, respectively. The obtained purities of 2′fucosyllactose on the sum of 2FL, diFL and lactose concentration for SEQ ID NO 01 or 02 ranged between 80% and 99.9% with the remaining carbohydrate (20% to 1%) mainly being lactose, which is the result of an incomplete conversion of lactose.

### Example 11: Enzymatic Production of 2′ Fucosyllactose

Another example provides the use of an enzyme with SEQ ID NO 01 and 02 of the present invention. These enzymes are produced in a cell-free expression system such as but not limited to the PURExpress system (NEB), or in a host organism such as but not limited to Escherichia coli or Saccharomyces cerevisiae, after which the above listed enzymes can be isolated and optionally further purified.

Each of the above enzyme extracts or purified enzymes are added to a reaction mixture together with GDP-fucose and a buffering component such as Tris-HCl or HEPES and either lactose or 2′FL. Said reaction mixture is then incubated at a certain temperature (for example 37° C.) for a certain amount of time (for example 24 hours), during which the lactose or 2′FL will be converted by the enzyme using GDP-fucose to 2′fucosyllactose and difucosyllactose, respectively, the latter reaction only when the 2-fucosyltransferase has side activity on 2′FL. The 2′fucosyllactose and di-fucosyllactose are then separated from the reaction mixture by methods known in the art. Further purification of the 2′FL and/or diFL can be performed if preferred. At the end of the reaction or after separation and/or purification, the production of 2′fucosyllactose and di-fucosyllactose is measured as described in Example 2.

### Example 12: 2′Fucosyllactose Production in a Bioreactor

A fed-batch fermentation process as described in Example 2 was performed wherein the lactose concentration in the culture medium was set at 120 g/L. The same process was performed with different 2′fucosyllactose production strains, as described in Example 2, containing an expression plasmid with the alpha-1,2-fucosyltransferase enzymes with SEQ ID NOs 01, 03, 04 or 71. Said lactose is converted during the process into 2′fucosyllactose until minor amounts of lactose is left. The final ratio lactose to 2′fucosyllactose may be manipulated during this process by stopping the process earlier (higher lactose to 2′fucosyllactose ratio) or later (lower lactose to 2′fucosyllactose ratio).[CS25] Also the final ratio diFL to 2′fucosyllactose may be manipulated similarly for strains expressing a 1,2-fucosyltransferase enzymes producing 2′FL and having side-activity forming diFL by stopping the process earlier (lower diFL to 2′fucosyllactose ratio) or later (higher diFL to 2′fucosyllactose ratio).[NL-I26][27]

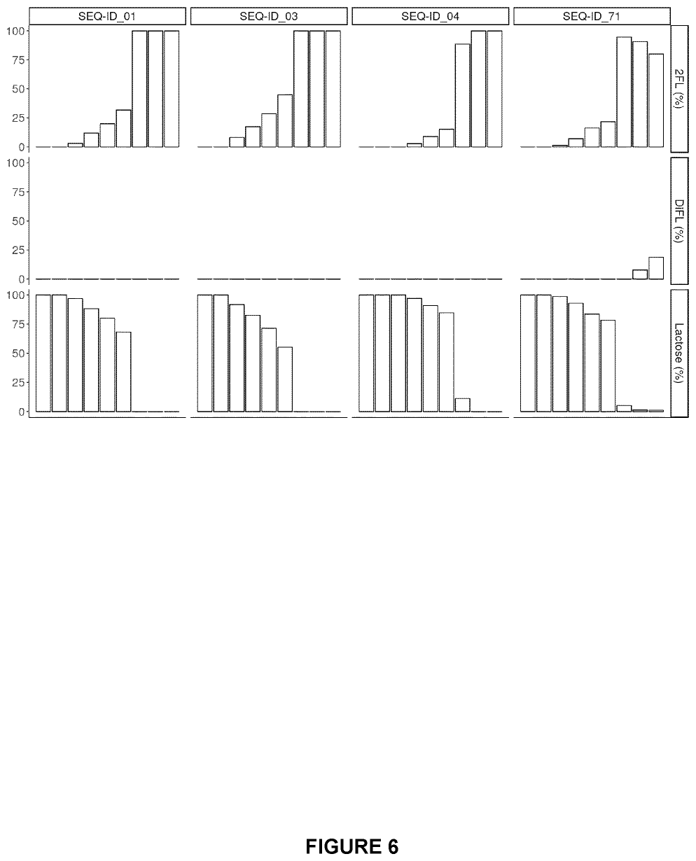

FIG. 6 shows the conversion of lactose into 2′fucosyllactose and diFL during these fermentations. The x-axis depicts various timepoints during the fermentation from start (100% lactose present) to finish (when no or almost no lactose is detected anymore). The y-axis depicts the percentages of lactose, 2′FL and diFL present in the fermentation broth, determined by dividing each sugar by the sum of the three sugars. At the end of the fermentations with the strains with SEQ ID NO 01, 03 or 04, a very high 2′FL purity (near 100%) could be reached. No lactose could be detected anymore and no diFL was formed, even when the lactose was completely depleted. In contrast, in a similar fermentation with a strain containing SEQ ID NO 71, small amounts of lactose remained at the end of the fermentation. Simultaneously, when the lactose concentration dropped below 5%, diFL started to appear in the fermentation broth (up to 19% of the total sugar concentration in this example).

It is thus clear that much higher 2′fucosyllactose purities can be obtained using the alpha-1,2-fucosyltransferase enzymes with SEQ ID NO 01, 03 or 04 compared to the enzyme with SEQ ID NO 71.

### Example 13: Enzymatic Production of 2′ Fucosyllactose and Di-Fucosyllactose

Another example provides the use of an enzyme with SEQ ID NO 01, 02, 03, 04 or 71 of the present invention. These enzymes are produced in Escherichia coli, after which the cells are harvested and lysed in Tris-HCl buffer using sonication. The soluble protein fraction is separated from the cell debris and used for downstream enzymatic reaction. Each of the above enzyme extracts are added to a reaction mixture together with GDP-L-fucose as the donor substrate (5 mM), a buffering component (Tris-HCl) and either lactose or 2′FL as the acceptor substrate (5 mM). Note that both the donor and acceptor substrates are present in excess, in concentrations higher than physiologically possible. Said reaction mixture is then incubated at 37° C. for 24 hours. The lactose, 2′fucosyllactose and di-fucosyllactose are then separated and detected using liquid chromatography as described in Example 2.

The experiment showed that using lactose as a substrate, the enzyme with SEQ ID NO 71 (HpFutC) was able to produce both 2′FL and diFL, while the enzymes with SEQ ID NO 01, 02, 03 and 04 only produced 2′FL and no diFL. Similarly, using 2′FL as a substrate, only the enzyme with SEQ ID NO 71 (HpFutC) was able to produce diFL, while no further conversion of 2′FL to diFL was observed for the enzymes with SEQ ID NOs 01, 02, 03 and 04. Thus, the enzyme with SEQ ID NO 71 (HpFutC) produced diFL as a side-product, while the other tested enzymes did not (or at least to a much lesser extent, below detection limit). While for strains with HpFutC, the in vivo produced diFL to 2′FL ratio could be kept low by limiting the GDP-L-fucose donor supply, this low GDP-fucose donor supply simultaneously limited the 2′FL productivity. For strains containing the enzymes with SEQ ID NOs 01, 02, 03 or 04 the in vivo GDP-L-fucose concentration supplied was irrelevant with respect to the diFL to 2′FL ratio and thus a much higher 2′FL productivity could be achieved.

1.-35. (canceled)

36. A method of producing α-1,2-fucosyllactose, the method comprising the steps of:
a) providing a polypeptide with α-1,2-fucosyltransferase activity and able to use lactose as an acceptor substrate, wherein the polypeptide
i) comprises a conserved domain G-Y-[F/Y]-Q-[N/S] (SEQ ID NO: 72) and a conserved domain (X, no K/V)XXXX[I/L]H[I/L]R[R/L]GD[F/Y](X, no C/M) (SEQ ID NO: 74), wherein X can be any distinct amino acid excluding a lysine and a valine residue from the first position of the domain and excluding a cysteine and a methionine residue from the last position of the domain, and/or
ii) is selected from the group consisting of:
i) any one of SEQ ID NOs: 1-7, 9-17, 19, 21, 22, 25, 26, 29, 33-41, 43-48, 50-58, 61-63, 66, 67, or 76,
ii) a polypeptide having 80% or more sequence identity to a full-length amino acid sequence of any one of SEQ ID NOs: 1-31, 33-63, 65-70, 75-78, or 79,
iii) a polypeptide of an allelic variant of a polypeptide of one of SEQ ID NOs: 1-31, 33-63, 65-70, 75-78, or 79,
iv) a polypeptide of an ortholog of a polypeptide of one of SEQ ID NOs: 1-31, 33-63, 65-70, 75-78, or 79, and
v) a functional fragment of a polypeptide of one of SEQ ID NOs: 1-31, 33-63, 65-70, 75-78, or 79,
b) contacting the polypeptide of step a) with a mixture comprising GDP-fucose as a donor substrate and lactose as an acceptor substrate, under conditions wherein the polypeptide catalyses a transfer of a fucose residue from the donor substrate to the acceptor substrate, thereby producing α-1,2-fucosyllactose,
c) optionally separating the α-1,2-fucosyllactose, and
d) optionally further comprising purifying α-1,2-fucosyllactose thus produced.

37. The method according to claim 36, wherein said polypeptide:
is selected from the group consisting of:
i) any one of SEQ ID NOs: 1-7, 9-31, 33-63, 65-67, 70, or 75-79,
ii) a polypeptide having 80% or more sequence identity to a full-length amino acid sequence of any one of SEQ ID NOs: 1-7, 9-31, 33-63, 65-67, 70, or 75-79,
iii) a polypeptide of an allelic variant of a polypeptide of one of SEQ ID NOs: 1-7, 9-31, 33-63, 65-67, 70, or 75-79,
iv) a polypeptide of an ortholog of a polypeptide of one of SEQ ID NOs: 1-7, 9-31, 33-63, 65-67, 70, or 75-79, and
v) a functional fragment of a polypeptide of one of SEQ ID NOs: 1-7, 9-31, 33-63, 65-67, 70, or 75-79,
or wherein said polypeptide:
i) comprises a conserved domain G-Y-[F/Y]-Q-[N/S] (SEQ ID NO: 72) and a conserved domain (X, no K/V)XXXX[I/L]H[I/L]R[R/L]GD[F/Y](X, no C/M) (SEQ ID NO: 74), wherein X can be any distinct amino acid excluding a lysine and a valine residue from the first position of the domain and excluding a cysteine and a methionine residue from the last position of the domain, and
ii) is selected from the group consisting of:
i) any one of SEQ ID NOs: 8, 32, 64, 68, or 69,
ii) a polypeptide having 80% or more sequence identity to a full-length amino acid sequence of any one of SEQ ID NOs: 8, 32, 64, 68, or 69,
iii) a polypeptide of an allelic variant of a polypeptide of one of SEQ ID NOs: 8, 32, 64, 68, or 69,
iv) a polypeptide of an ortholog of a polypeptide of one of SEQ ID NOs: 8, 32, 64, 68, or 69, and
v) a functional fragment of a polypeptide of one of SEQ ID NOs: 8, 32, 64, 68, or 69.

38. The method according to claim 36, which results in a diFL concentration to 2′fucosyllactose concentration ratio of less than 1:5.

39. The method according to claim 36, wherein the polypeptide is provided in a cell free system.

40. The method according to claim 36, wherein the polypeptide is produced by a host cell comprising a polynucleotide encoding the polypeptide.

41. The method according to claim 36, wherein the GDP-fucose and/or lactose is provided by a cell producing GDP-fucose and/or lactose.

42. The method according to claim 40, the method comprising:
growing a host cell expressing the polypeptide under suitable nutrient conditions permissive for producing the α-1,2-fucosyllactose, and permissive for expression of the polypeptide;
providing simultaneously or subsequently a donor substrate GDP-fucose and an acceptor substrate lactose, in order for the α-1,2-fucosyltransferase polypeptide to catalyze transfer of a fucose residue from GDP-fucose to lactose, thereby producing α-1,2-fucosyllactose;
optionally separating the α-1,2-fucosyllactose from the host cell or medium of its growth;
and optionally further comprising purifying α-1,2-fucosyllactose thus produced.

43. The method according to claim 42, wherein the host cell is transformed or transfected to express an exogenous polypeptide with alpha-1,2-fucosyltransferase activity and able to use lactose as an acceptor substrate.

44. The method according to claim 40, wherein the GDP-fucose and/or lactose is provided by an enzyme simultaneously expressed in the host cell or by the host cell's metabolism.

45. The method according to claim 36, wherein the method further comprises at least one of the following steps:
i) adding, in a continuous manner, a lactose feed to a culture medium in a reactor having a volume, wherein the lactose feed comprises at least 50 grams of lactose per liter of initial reactor volume, wherein the total reactor volume is from 250 mL (milliliter) to 10,000 m³ (cubic meter) so that the culture medium's final volume is not more than three-fold of the volume of the culture medium before adding the lactose feed;
ii) adding, in a continuous manner, a GDP-fucose feed to a culture medium, wherein the GDP-fucose feed has a concentration enabling a host cell to synthesize 2′-fucosyllactose with a diFL concentration to 2′fucosyllactose concentration ratio of less than 1:5, so that the culture medium's final volume is not more than three-fold of the volume of the culture medium before adding the GDP-fucose feed;
iii) adding, in a continuous manner, a carbon-based substrate feed to a culture medium, wherein the carbon-based substrate feed is at a concentration enabling a host cell to synthesize GDP-fucose at a concentration for 2′fucosyllactose synthesis with a diFL concentration to 2′fucosyllactose concentration ratio of less than 1:5;
iv) adding to a culture medium a carbon-based substrate feed at a concentration enabling a host cell to internally synthesize lactose at a concentration allowing 2′fucosyllactose synthesis;
v) adding, in a continuous manner, a lactose feed, a GDP-fucose feed, and/or a carbon-based substrate feed to a culture medium over the course of 1 day, 2 days, 3 days, 4 days, or 5 days by means of at least one feeding solution; and/or
vi) adding, in a continuous manner, a lactose feed to culture medium over the course of 1 day, 2 days, 3 days, 4 days, or 5 days by means of a lactose feeding solution, wherein the concentration of lactose feeding solution is at least 50 g/L, wherein the pH of the lactose feeding solution is between 3 and 7, and wherein the temperature of the lactose feeding solution is kept between 20° C. and 80° C.,
wherein the method results in a 2′-fucosyllactose concentration of at least 50 g/L in the culture medium's final volume.

46. A host cell genetically modified to produce alpha-1,2-fucosyllactose, the host cell comprising:
at least one polynucleotide encoding an enzyme for α-1,2-fucosyllactose synthesis;
wherein the host cell expresses a polypeptide with α-1,2-fucosyltransferase activity and is able to use lactose as an acceptor substrate, and
wherein the polypeptide:
i) comprises a conserved domain G-Y-[F/Y]-Q-[N/S] (SEQ ID NO: 72) and a conserved domain (X, no K/V)XXXX[I/L]H[I/L]R[R/L]GD[F/Y](X, no C/M) (SEQ ID NO: 74), wherein X can be any distinct amino acid excluding a lysine and a valine residue from the first position of the domain and excluding a cysteine and a methionine residue from the last position of the domain; and/or,
ii) is selected from the group consisting of:
a) any one of SEQ ID NOs: 1-7, 9-17, 19, 21, 22, 25, 26, 29, 33-41, 43-48, 50-58, 61-63, 66, 67, or 76;
b) a polypeptide having 80% or more sequence identity to a full-length amino acid sequence of any one of SEQ ID NOs: 1-31, 33-63, 65-70, 75-78, or 79;
c) a polypeptide of an allelic variant of a polypeptide of one of SEQ ID NOs: 1-31, 33-63, 65-70, 75-78 or 79;
d) an ortholog of a polypeptide of any of SEQ ID NOs: 1-31, 33-63, 65-70, 75-78 or 79; and
e) a functional fragment of a polypeptide of any one of SEQ ID NOs: 1-31, 33-63, 65-70, 75-78 or 79.

47. The host cell of claim 46, wherein said polypeptide
is selected from the group consisting of:
i) any one of SEQ ID NOs: 1-7, 9-31, 33-63, 65-67, 70, or 75-79,
ii) a polypeptide having 80% or more sequence identity to a full-length amino acid sequence of any one of SEQ ID NOs: 1-7, 9-31, 33-63, 65-67, 70, or 75-79,
iii) a polypeptide of an allelic variant of a polypeptide of one of SEQ ID NOs: 1-7, 9-31, 33-63, 65-67, 70, or 75-79,
iv) a polypeptide of an ortholog of a polypeptide of one of SEQ ID NOs: 1-7, 9-31, 33-63, 65-67, 70, or 75-79, and
v) a functional fragment of a polypeptide of one of SEQ ID NOs: 1-7, 9-31, 33-63, 65-67, 70, or 75-79,
or wherein said polypeptide:
i) comprises a conserved domain G-Y-[F/Y]-Q-[N/S] (SEQ ID NO: 72) and a conserved domain (X, no K/V)XXXX[I/L]H[I/L]R[R/L]GD[F/Y](X, no C/M) (SEQ ID NO: 74), wherein X can be any distinct amino acid excluding a lysine and a valine residue from the first position of the domain and excluding a cysteine and a methionine residue from the last position of the domain, and
ii) is selected from the group consisting of:
i) any one of SEQ ID NOs: 8, 32, 64, 68, or 69,
ii) a polypeptide having 80% or more sequence identity to a full-length amino acid sequence of any one of SEQ ID NOs: 8, 32, 64, 68, or 69,
iii) a polypeptide of an allelic variant of a polypeptide of one of SEQ ID NOs: 8, 32, 64, 68, or 69,
iv) a polypeptide of an ortholog of a polypeptide of one of SEQ ID NOs: 8, 32, 64, 68, or 69, and
v) a functional fragment of a polypeptide of one of SEQ ID NOs: 8, 32, 64, 68, or 69.

48. The host cell of claim 46, wherein the host cell comprises:
i) a nucleotide sequence comprising a polynucleotide encoding the polypeptide with lactose binding α-1,2-fucosyltransferase activity, wherein the nucleotide sequence is foreign to the host cell and is integrated into the host cell's genome, or
ii) a vector comprising a polynucleotide encoding the polypeptide, wherein the polynucleotide is operably linked to control sequences recognized by a host cell transformed with the vector.

49. The host cell of claim 46, wherein the cell is selected from the group consisting of microorganism, plant cell and animal cell.

50. The host cell of claim 46, wherein the cell is a microorganism that heterologously expresses said α-1,2-fucosyltransferase polypeptide.

51. The host cell of claim 45, wherein the polynucleotide encoding the polypeptide with lactose binding alpha-1,2-fucosyltransferase activity is adapted to the codon usage of the respective host cell.

52. A method of using the host cell of claim 45 to produce α-1,2-fucosyllactose, the method comprising:
cultivating the host cell in a medium under conditions permissive for producing α-1,2-fucosyllactose,
optionally, separating the produced α-1,2-fucosyllactose from the cultivation, and
optionally further comprising purifying α-1,2-fucosyllactose thus produced.

53. The method according to claim 36, wherein the separation comprises at least one of clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography, gel filtration, and ligand exchange chromatography.

54. The method according to claim 36, further comprising purifying α-1,2-fucosyllactose thus produced, wherein purification of alpha-1,2-fucosyllactose comprises at least one of: use of activated charcoal or carbon, use of charcoal, nanofiltration, ultrafiltration or ion exchange, use of alcohols, use of aqueous alcohol mixtures, crystallization, evaporation, precipitation, drying, spray drying, or lyophilisation.

55. The method according to claim 36, having a lactose concentration in the culture medium of between 50 g/L and 150 g/L.

56. The method according to claim 36, having a final concentration of 2′-fucosyllactose between 70 g/L and 200 g/L.
